(19) United States
(12) Patent Application Publication
(10) Pub. No.: US 2025/0218547 A1
(43) Pub. Date: Jul. 3, 2025

(54) LEARNED BREEDING STRATEGIES

(71) Applicant: PIONEER HI-BRED INTERNATIONAL, INC., JOHNSTON, IA (US)

(72) Inventors: RILEY MITCHELL MCDOWELL, JOHNSTON, IA (US); ELI RODGERS-MELNICK, JOHNSTON, IA (US); DANIEL SALMON, STANWOOD, WA (US)

(73) Assignee: PIONEER HI-BRED INTERNATIONAL, INC., JOHNSTON, IA (US)

(21) Appl. No.: 18/851,769

(22) PCT Filed: Mar. 28, 2023
(86) PCT No.: PCT/US2023/065049

§ 371 Date: Sep. 27, 2024

Related U.S. Application Data
(60) Provisional application No. 63/362,052, filed on Mar. 29, 2022.

(51) Int. Cl. G16B 40/20; G16B 20/40

(52) U.S. Cl. CPC G16B 40/20; G16B 20/40

(57) ABSTRACT

Systems and methods that use models in plant breeding advancement decisions are provided herein. Also provided are systems and methods that utilize ensembles to generate advancement scores for candidate plant genotypes for advancement. Also provided herein are systems and methods for use in producing plants, including plants from doubled haploid embryos, inbreds, and hybrids.

### CROSS-REFERENCE TO RELATED APPLICATIONS

This application claims priority to U.S. Provisional Application No. 63/362,052 filed Mar. 29, 2022, which is hereby incorporated by reference in its entirety.

### FIELD

The disclosure relates to methods and systems for use in or with plant breeding and plant breeding advancement and the production of plants.

### BACKGROUND

The contribution of plant breeding to agricultural productivity continues to grow at an astronomical rate as plant breeders have been adept at assimilating and integrating information from extensive potential lines and applying advanced breeding approaches to create a breeding pipeline that has continuous population improvement and delivers valued products for farmers, end-users, and consumers.

### SUMMARY

Disclosed herein are computer-implemented methods for use in plant breeding. The methods may include (a) receiving input data including data from candidate plant genotypes being considered for advancement through a computing device, (b) inputting candidate data including data from candidate plant genotypes being considered for advancement into the ensemble of the at least two trained machine learning models, where the at least two trained machine learning models have been trained to learn a likelihood of advancement of a plant, and (c) generating by the ensemble an advancement score for each candidate plant genotype. The methods may also include training the ensemble by (a) receiving, through one or more computing devices, at least one training data set including data from a breeder's selections of plants for advancement, (b) inputting the data from the at least one training data set into an ensemble of at least two machine learning models, (c) training the ensemble of the at least two machine learning models to learn a likelihood of advancement of a plant genotype from the training data set, (d) inputting candidate data including data from candidate plant genotypes being considered for advancement into the ensemble of the at least two trained machine learning models, and (e) generating by the ensemble an advancement score for each candidate plant genotype.

In some examples, the computer-implemented methods for use in plant breeding may include (a) inputting into a pre-trained deep learning model in a computing device a plurality of candidate plant genotypes that a breeding target environment is considering along with the breeding target environment token that is considering the plurality of candidate plant genotypes to generate an advancement score for each plant genotype.

Also disclosed are computer readable mediums having stored thereon instructions to provide candidate plant genotypes recommendations, when executed by a processor (or computing device), cause the processor to perform the steps of the computer-implemented methods.

Also disclosed herein are systems for use in plant breeding that include (a) one or more servers, each of the one or more server storing plant data, and (b) a computing device communicatively coupled to the one or more servers, the computing device including: (1) a memory, and (2) one or more processors configured to perform operations to: (a) obtain data from a plurality of candidate plant genotypes, and (b) generate an advancement score for each candidate plant genotype from the plurality of candidate plant genotypes using an ensemble of at least two trained machine learning models.

In some examples disclosed herein are systems for use in plant breeding that include (a) one or more servers, each of the one or more server storing plant data, and (b) a computing device communicatively coupled to the one or more servers, the computing device including: (1) a memory, and (2) one or more processors configured to perform operations including: (a) receive into a pretrained deep learning model a plurality of candidate plant genotypes that a breeding target environment is considering along with a breeding target environment token that is considering a plurality of candidate plant genotypes to generate an advancement score for each candidate plant genotype.

Also provided herein are computer-implemented methods for generating a representation for a plant genotype for one or more breeder's notes. In some examples, the methods include (a) receiving by a tokenizer implementing a tokenization scheme for a constructed vocabulary one or more breeder's notes, where the one or more breeder's notes include one or more word parts, (b) assigning each word part of the one or more breeder's notes a token, (c) assigning each breeder its own unique token, (d) receiving by a deep learning model implementing self-attention in a computing device one or more pairings of breeder and breeder's notes, where the breeders are tokenized, and the breeder's notes include one or more word parts that have been encoded into tokens using a constructed vocabulary, (e) converting by an embedding layer each token in the input to a unique token embedding corresponding to that token, (f) pretraining the deep learning model to learn structural language patterns in the breeder notes, relationships of notes to breeders, and how notes co-occur with one another among plant genotypes, the pretraining constituting a masked language modeling task including: (1) performing selection of one or more tokens to be evaluated by a loss function following an output layer, (2) generating replacement of one or more of the selected input token embeddings from (1) with either an alternative token embedding selected from a tokenizer vocabulary or a token embedding representing the masked state, (3) generating by the deep learning model a prediction of the true token for each input token, (4) evaluating the loss function of the predicted tokens with respect to their true values for those tokens selected in (1), (5) adjusting the weights of the token embeddings, the deep learning self-attention model, and a predictive output layer of the tokens to reduce the evaluated loss, (6) reiterating steps (1)-(5) until convergence of the loss to a desired value, and (g) inputting a plurality of pairs of breeder tokens and breeder note tokens into the pre-trained deep learning model to generate a vector that corresponds to a unified embedding representation of a plurality of breeder notes for a particular plant genotype.

Also provided herein are systems for use in plant breeding that include: (a) one or more servers, each of the one or more server storing plant data, and (b) a computing device communicatively coupled to the one or more servers, the computing device including: (1) a memory, and (2) one or more processors configured to perform operations including: (a) obtain one or more breeder's notes, where the breeder's notes include one or more word parts that have been encoded into tokens using a constructed vocabulary, (b) assign each breeder its own unique token, (c) pretrain a deep learning model to learn structural language patterns in the breeder notes, relationships of notes to breeders, and how notes co-occur with one another among plant genotypes, (1) mask and/or randomly replace a plurality of selected breeder tokens and breeder note tokens, (2) predict a true token for each input breeder token and breeder note token, (3) evaluate a loss function of the predicted tokens with respect to their true values, (4) adjust the weights of the token embeddings, the self-attention model, and the predictive output layer of the tokens to reduce evaluated loss, (d) receive a plurality of pairs of breeder tokens and breeder note tokens into the pre-trained deep learning model, and (e) generate a vector that corresponds to a unified embedding representation of a plurality of breeder notes for a particular plant genotype.

The methods and systems disclosed herein may be used on or with any plant genotype or candidate plant genotype. The plant genotype or candidate plant genotype may be a monocot or dicot plant. The methods and systems disclosed herein may be used for or with breeding advancement and the selection and production of plants.

### BRIEF DESCRIPTION OF THE DRAWINGS

FIG. 1. is a block diagram illustrating an exemplary computer system including a server and a computing device according to an embodiment as disclosed herein.

FIG. 2. is a schematic illustrating the input (training data) and output (learned breeding strategy) in one embodiment of training three different machine learning models to learn various breeding strategies.

FIG. 3. is a schematic illustrating the input (candidate data) and output (combined advancement score for a candidate plant genotype) in one embodiment of using learned breeding strategies using any number of established machine learning models.

FIG. 4. is a schematic illustrating the input (candidate data) and output (combined advancement score for a candidate plant genotype) in one embodiment of using learned breeding strategies using two established machine learning models.

FIG. 5. is a schematic illustrating the input (candidate data) and output (combined adjusted advancement score for a candidate plant genotype) in one embodiment of using learned breeding strategies using two established machine learning models and one penalty assessing established machine learning model.

FIG. 6 is a schematic illustrating the input (candidate data) and output (combined adjusted advancement score for a candidate plant genotype) in one embodiment of using learned breeding strategies using two established machine learning models and one penalty assessing established machine learning model.

FIG. 7. is a schematic illustrating the input (candidate data) and output (combined adjusted advancement score for a candidate plant genotype) in one embodiment of using learned breeding strategies using two established machine learning models and one penalty assessing established machine learning model.

FIG. 8A is an illustration of a 2-trait retrospective index model in one embodiment of a machine learning model in Example 1. Black dots represent selected lines and gray X's represent unselected (dropped) lines. This breeder prefers to select lines that have high yield (YIELD) for a given level of grain moisture (MST). In two dimensions, the retrospective index may be conceptualized as a 2-dimensional regression residual. With more than two traits, the index may be conceptualized as a residual from a multi-trait hyperplane.

FIG. 8B is an illustration of advancement scores from a 2-trait retrospective index model in one embodiment of a machine learning model in Example 1. Larger sized black dots indicate higher levels of predicted likelihood of advancement.

FIG. 9A is an illustration of a 2-trait multivariate Gaussian model in one embodiment of a machine learning model in Example 2. Black dots represent selected lines and gray X's represent unselected (dropped) lines, with gray contour regions depicting probability density. This breeder does not select lines outside a range of acceptable trait values. In two dimensions, the multivariate gaussian index may be conceptualized as a 2-variable joint probability. With k traits, a k-dimensional Gaussian is fitted.

FIG. 9B is an illustration of advancement scores from a two-variable multivariate Gaussian index model in one embodiment of a machine learning model in Example 2. Larger sized black dots indicate higher levels of predicted likelihood of advancement. Note that lines far outside the breeder's typical trait values are given low predicted merit likelihood of advancement.

FIG. 10A is an illustration of a maturity distribution model in one embodiment of a machine learning model in Example 3. Black dots represent selected lines and gray X's represent unselected (dropped) lines, with a probability distribution overlaid on the harvest grain moisture (MST) axis. Grain moisture is highly correlated with maturity, and this breeder limits selections to lines with maturity appropriate for their target market. Note that the lines with lowest and highest MST are not selected and fall in the tails of the probability distribution.

FIG. 10B is an illustration of advancement scores from a maturity distribution model in one embodiment of a machine learning model in Example 3. Larger sized black dots indicate higher levels of predicted likelihood of advancement. Note that only the MST trait is considered by this component model.

FIG. 11A is an illustration of a complete learned breeding strategy on a decision dataset in one embodiment of an ensemble in Example 4. Larger sized black dots indicate higher levels of predicted likelihood of advancement. The retrospective index and multivariate Gaussian component models included 31 agronomically relevant traits.

FIG. 11B is an illustration of a learned breeding strategy utilized with a large germplasm library in one embodiment of an ensemble in Example 4. Larger sized black dots represent lines that are predicted to be of interest to the breeder based on their learned breeding strategy from the decision dataset.

FIGS. 12A and 12B show excerpts from a table of candidate lines scored using 13 learned breeding strategies from Example 5. Each row is a unique candidate. Each column contains standard-normal transformed scores from a different learned breeding strategy. The scores indicate a likelihood of advancement, wherein a higher score indicates a candidate line with a higher likelihood of advancement compared to a candidate line with a lower score. The rightmost column is an average of the scores from the 13 learned breeding strategies rescaled to have mean 100 and variance 100.

FIG. 12A shows a table sorted to show candidate lines with the largest average scores from the learned breeding strategy from Example 5. Most cells contain a positive value greater than 2, indicating the line scored a standard deviation or more above average for that column's learned strategy. The average scores are mostly above 120, indicating these lines also performed at least 2 standard deviations above the average learned breeding strategy. Together these indicate strong agreement between breeder's strategies to select the best lines.

FIG. 12B shows a table sorted to show candidate lines with the smallest average scores from the learned breeding strategies from Example 5. The cells are mostly less than negative 2 and the average scores are mostly less than 80, indicating these lines predicted about 2 standard deviations below average for the breeder's strategies, as well as for the average strategy, again indicating strong agreement between strategies on what constitutes the least valuable lines.

FIG. 13 shows a comparison of the selected lines from a simulated learned breeding strategy with (B) and without (A) the imposition of a coancestry penalty. Each point represents a candidate genotype for selection, and the dotted line indicates the constant selection threshold at an intensity of 50% under a purely yield and moisture-based learned breeding strategy. Without the coancestry penalty, only points above this threshold are selected. After the imposition of the coancestry penalty with a penalty weight of 0.5, several of the genotypes with LBS trait values above the target threshold are replace with those slightly below the threshold due to the former having high average coancestry with the other selected entries.

FIG. 14 shows the impact of increasing the weight of the coancestry penalty from 0 (LBS) to 10 (LBS_lambda_10). FIG. 14A shows the pairwise Pearson correlations between the advancement scores with different penalty weights, showing that these values become less correlated with one another as the penalty weights are moved further apart. Effectively, the penalty weight controls the trade-off between the relative impact of pure trait performance versus diversity on the final advancement score. FIG. 14B shows the impact of the coancestry penalty on a subset of genotypes as a function of average coancestry to the selected set and the magnitude of the penalty weight. As the penalty increases from 0, rank shifts occur in relative scores, with lines having high coancestry to the selected set rapidly decreasing in score and more diverse lines increasing.

FIG. 15 is a schematic of one example of a multilayer transformer-based model encoder module for use in deep learning predictions. In this instance, genotypes-encoded based on their SNP markers—are embedded into d-dimensional tokens alongside a d-dimensional token corresponding to the breeding target environment. These are fed forward through L layers of transformer encoder modules, followed by an output layer producing values in the unit interval. Pretraining tasks of genotype-genotype context (GGC) and genotype-breeder context (GBC) are defined. In the GGC task, the network is trained to predict whether a minority M genotypes correspond to the same location and experiment as the majority N-M genotypes, while the GBC task trains the network to predict whether the majority N-M genotypes belong to the given breeding target environment.

FIG. 16 is a schematic of one example of a multilayer transformer-based model encoder module for use in the encoding of breeder notes for downstream use informing learned breeding strategies. M pairs of breeders and breeder notes are input into the model. Each breeder is represented as a single token, while each breeder note is represented as a set of tokens taken from a vocabulary developed through byte pair encoding (BPE) of all breeder notes. Tokens representing separation-<September>-serve as indicators of distinct breeder, breeder note pairs. Tokens are embedded as d-dimensional vectors, to which spatial encodings are added to preserve ordering information through all transformer layers. Pre-training task outputs include V-dimensional multinomial vectors for each input slot, wherein V denotes the cardinality of the full token vocabulary. The multinomial outputs are used during the masked-language-modelling (MLM) task wherein the task is to correctly predict sets of tokens that are either masked or randomly replaced in the input. A single output is provided at the slot corresponding to the <CLS> token input, and this may be used for the next-sentence-prediction (NSP) task, wherein the loss is calculated based on the ability of the network to correct discern whether notes are matched with the correct breeder and whether two notes are given with respect to the same genotype.

### DETAILED DESCRIPTION

It is to be understood that this invention is not limited to particular embodiments, which can, of course, vary. It is also to be understood that the terminology used herein is for the purpose of describing particular embodiments only, and is not intended to be limiting. Further, all publications referred to herein are each incorporated by reference for the purpose cited to the same extent as if each was specifically and individually indicated to be incorporated by reference herein.

Every year, breeders evaluate lines and make decisions regarding what lines should be selected, crossed, and advanced to create a product or variety in their pipeline that has certain desirable traits or properties for a particular market or geography. Breeders may make these decisions based on any number of criteria, for example, personal experience, genetics, selection pressure to relevant traits for their geography and market conditions, and success and failures within their target population of environments. Further, the advancement selection process is often tedious and manual-intensive, for example, involving the construction or recreation from scratch of every previous decision using spreadsheets.

As technology evolves, breeders are facing even more options to consider for advancement stemming from larger numbers of candidate lines, numerous options for relevant trait predictions, tens of thousands of candidate lines with predicted genetic values from which to choose, and numerous predicted traits relative to selection pressure to choose among. By the time a breeder combines this volume of information together to make an effective advancement decision, it may be hard to share the breeding strategy with others in the breeding program and document it in a meaningful way.

To facilitate the understanding of individual or multiple breeder advancement decision making, for example, breeders targeting similar product concepts, related germplasm, and/or similar product maturity, the methods and systems described herein enable the machine learning of a breeder's strategy for selecting lines for advancement, for example, the likelihood that a candidate would be selected for advancement by a particular breeder or breeders, and/or the machine learning of a breeder's strategy. In some examples, the methods and systems described herein enable the machine learning of a breeder's strategy for selecting lines for discarding lines from further advancement consideration, for example, the likelihood that a candidate would be dropped from further advancement consideration by a particular breeder or breeders. As used herein, the term “likelihood” also refers to the propensity or probability that an event will occur, e.g. the likelihood that a candidate would be selected for advancement by a particular breeder or breeders, and/or the machine learning of a breeder's strategy. The modeled (learned) breeding strategies may be utilized with new, and potentially larger, datasets. In this way, use of the learned breeding strategies enables the reproduction of historical selection decisions if desired and/or the application to future datasets to make advancement recommendations for lines that are in keeping with a breeder's selection strategy and in a reproducible, consistent way. As demonstrated in Example 5, the candidates recommended for advancement using the learned breeding strategies were consistent with the actual advancement selection decisions when utilized across multiple years of decision datasets for the same breeder. Further, because an advancement score is generated for each candidate in a dataset, each candidate is able to be robustly quantified in terms of its interest to a breeder(s) or breeding program.

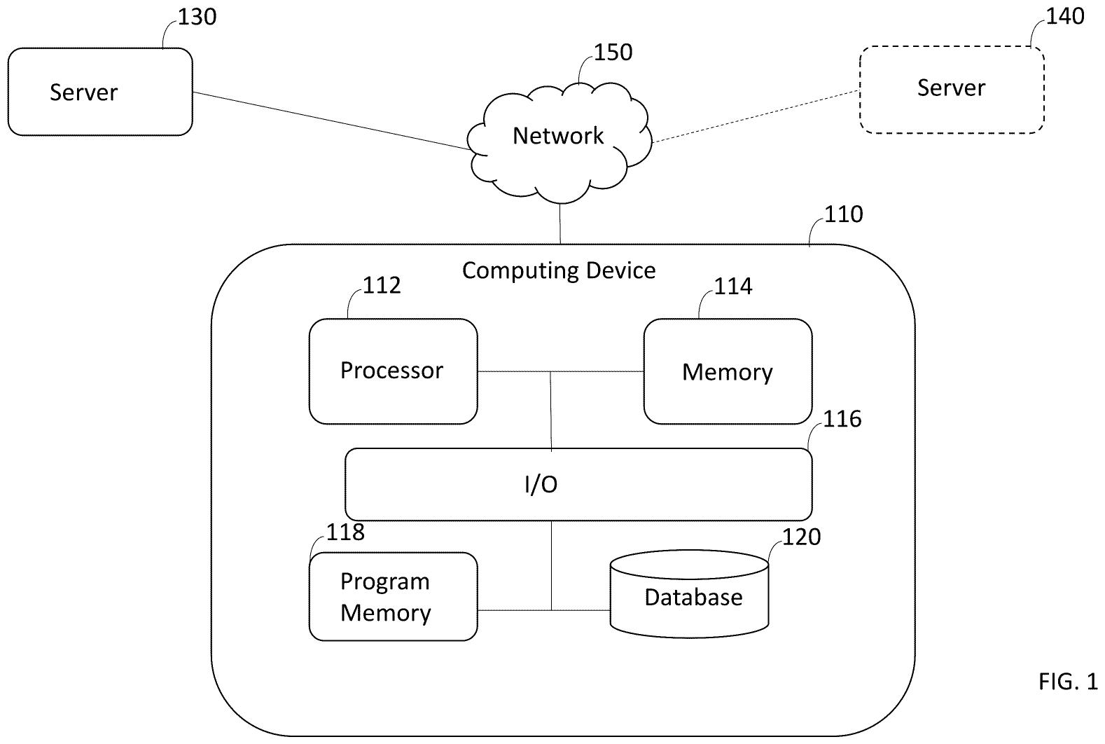

Referring to FIG. 1, a block diagram of a computer system 100 for learning breeding strategies and creating recommendations for plants for advancement is shown. To do so, the system 100 may include a computing device 110 and a server 130 that is associated with a computer system. The system 100 may further include one or more servers 140 that are associated with other computer systems such that the computing device 110 may communicate with different computer systems running different platforms. However, it should be appreciated that, in some embodiments, a single server (e.g., a server 130) may run multiple platforms. The computing device 110 is communicatively coupled to the one or more servers 130, 140 via a network 150 (e.g., a local area network (LAN), a wide area network (WAN), a personal area network (PAN), the Internet, etc.).

In use, the computing device 110 may make recommendations of plants for advancement by using an ensemble of at least two machine learning models, a deep learning model, or an ensemble consisting of ML and deep learning models to generate an advancement score for each candidate plant genotype. The advancement score may be raw or standard-normal transformed. More specifically, the computing device 110 may obtain data, such as training plant datasets or candidate plant datasets, stored in a database 120 and/or input by a user. For example, in the context of recommending plants for advancement, an ensemble or individual deep learning model may be trained to learn breeding strategies for a particular user, e.g. a breeder or multiple breeders, from one or more training datasets. The machine learning models are trained to learn a breeder's strategy and, in some embodiments, the trained models use the learned breeding strategy with a candidate's data and quantify the likelihood of the candidate's advancement.

In some examples, the training dataset may include plant genotypes that were selected for advancement in a breeding program, plants that were considered but ultimately not selected for advancement in a breeding program, or both. In some instances the set or subset of plants used to train the machine learning models may depend on the type of model being used. For example, as shown in Example 1, where the machine learning model creates a specific selection index value when it learns a breeding strategy, the input dataset uses data from all plants that were considered in advancement decisions regardless of whether the plants were selected for advancement.

The one or more training datasets may include but are not limited to data representations of genotypes, phenotypes, mean locus effects (MLE) data, breeder's field notes, environmental data, predicted genetic value, pedigree information, co-ancestry information, or combinations thereof. For example, genotypic data may include genome sequence information selected from the group consisting of SNP, QTL, RNA-seq, short read genomic sequencing, marker data, long read genome sequence information, methylation status, gene expression values, indels, haplotypes, and combinations thereof. In some aspects, the genotypic data includes a collection of genotypic markers, such as genome-wide markers, or single nucleotide polymorphisms (SNPs). Phenotypic data may include but is not limited to predicted yield gain, root lodging, stalk lodging, brittle snap, ear height, grain moisture, plant height, disease resistance, drought tolerance, or a combination thereof. Phenotypic data may include but is not limited to a molecular phenotype including but not limited to gene expression, chromatin accessibility, DNA methylation, histone modifications, recombination hotspots, genomic landing locations for transgenes, transcription factor binding status, or a combination thereof. In some examples, the phenotypes include those that are imputed rather than directly measured. Mean locus effects data may include values representing the average effect of loci in the genome of lines within their geography for a particular trait or traits and used to predict additive genetic value of lines. Exemplary, non-limiting traits include yield, disease resistance, agronomic traits, abiotic traits, kernel composition (including, but not limited to protein, oil, and/or starch composition), insect resistance, fertility, silage, and morphological traits, such as but not limited to days to pollen shed, days to silking, leaf extension rate, chlorophyll content, leaf temperature, stand, seedling vigor, internode length, plant height, leaf number, leaf area, leaf angle, tillering, brace roots, stay green, stalk lodging, root lodging, plant health, barreness/prolificacy, green snap, pest resistance, number of kernels per row on the ear, number of rows of kernels on the ear, kernel abortion, kernel weight, kernel size, kernel density and physical grain quality, shatter resistance, and uniformity.

Breeder's field notes may include but are not limited to general field appearance, parentability, plot quality, environment quality, opportunity traits, such as disease presence and lodging, and the like. Environmental data may include but is not limited to data for soil properties, irrigation, precipitation, temperature, solar radiation, plant population density, planting date, nutrient application, seed- or soil-applied agricultural biologicals, crop rotations, and targeted in-season crop protection agent. In some examples, the environmental data comes from a field or greenhouse.

In some examples, the data comes from plants grown in a field, greenhouse, or laboratory. In some examples, the data may be obtained from any suitable plants or parts thereof, for example, cells, seeds, leaves, immature plants, seedlings, or mature plants. In some examples, the plants are inbred plants, hybrid plants, doubled haploid plants, including but not limited to F1 or F2 doubled haploid plants, offspring or progeny thereof, including those from in silico crosses, or any combination of one or more of the foregoing. Any monocot or dicot plant genotype may used with the methods and systems provided herein, including but not limited to a soybean, maize, sorghum, cotton, canola, sunflower, rice, wheat, sugarcane, alfalfa tobacco, barley, cassava, peanuts, millet, oil palm, potatoes, rye, or sugar beet plant.

The ensemble of two or machine learning models, individual deep learning model, or ensemble of machine learning and deep learning models may be trained to learn a breeding strategy from training datasets regarding which plants have a higher or greater likelihood of being selected for advancement and/or dropped from advancement. In some examples, the one or more training datasets may be selected based on the user, environmental conditions, geographic regions, candidate genotypes, candidate phenotypes, genetic values obtained from MLE, and/or additional considerations or combinations thereof. In some examples, the training datasets may be further selected based on additional considerations, for example, specific years, genetic clusters, including without limitation heterotic groups and maturity ranges.

In general, the computing device 110 may include any existing or future devices capable of training a machine learning model. For example, the computing device may be, but not limited to, a computer, a notebook, a laptop, a mobile device, a smartphone, a tablet, wearable, smart glasses, or any other suitable computing device that is capable of communicating with the server 130.

The computing device 110 includes a processor 112, a memory 114, an input/output (I/O) controller 116 (e.g., a network transceiver), a memory unit 118, and a database 120, all of which may be interconnected via one or more address/data bus. It should be appreciated that although only one processor 112 is shown, the computing device 110 may include multiple processors. Although the I/O controller 116 is shown as a single block, it should be appreciated that the I/O controller 116 may include a number of different types of I/O components (e.g., a display, a user interface (e.g., a display screen, a touchscreen, a keyboard), a speaker, and a microphone).

The processor 112 as disclosed herein may be any electronic device that is capable of processing data, for example a central processing unit (CPU), a graphics processing unit (GPU), a tensor processing unit (TPU), a system on a chip (SoC), or any other suitable type of processor. It should be appreciated that the various operations of example methods described herein (i.e., performed by the computing device 110) may be performed by one or more processors 112. The memory 114 may be a random-access memory (RAM), read-only memory (ROM), a flash memory, or any other suitable type of memory that enables storage of data such as instruction codes that the processor 112 needs to access in order to implement any method as disclosed herein. It should be appreciated that, in some embodiments, the computing device 110 may be a computing device or a plurality of computing devices with distributed processing.

As used herein, the term “database” may refer to a single database or other structured data storage, or to a collection of two or more different databases or structured data storage components. In the illustrative embodiment, the database 120 is part of the computing device 110. In some embodiments, the computing device 110 may access the database 120 via a network such as network 150. The database 120 may store data (e.g., input, output, intermediary data) used for generating recommendations of plants for advancement. For example, the data may include genotypic data, such as single nucleotide polymorphisms (SNPs), genetic markers, haplotype, sequence information, phenotypic data, mean locus effects (MLE) data, breeder's field notes, environmental data, predicted genetic values, pedigree information, co-ancestry information, or combinations thereof that are obtained from one or more servers 130, 140.

The computing device 110 may further include a number of software applications stored in a memory unit 118, which may be called a program memory. The various software applications on the computing device 110 may include specific programs, routines, or scripts for performing processing functions associated with the methods described herein. Additionally or alternatively, the various software applications on the computing device 110 may include general-purpose software applications for data processing, database management, data analysis, network communication, web server operation, or other functions described herein or typically performed by a server. The various software applications may be executed on the same computer processor or on different computer processors. Additionally, or alternatively, the software applications may interact with various hardware modules that may be installed within or connected to the computing device 110. Such modules may implement part of or all of the various exemplary method functions discussed herein or other related embodiments.

Although only one computing device 110 is shown in FIG. 1, the server 130, 140 is capable of communicating with multiple computing devices similar to the computing device 110. Although not shown in FIG. 1, similar to the computing device 110, the server 130, 140 also includes a processor (e.g., a microprocessor, a microcontroller), a memory, and an input/output (I/O) controller (e.g., a network transceiver). The server 130, 140 may be a single server or a plurality of servers with distributed processing. The server 130, 140 may receive data from and/or transmit data to the computing device 110.

The network 150 is any suitable type of computer network that functionally couples at least one computing device 110 with the server 130, 140. The network 150 may include a proprietary network, a secure public internet, a virtual private network and/or one or more other types of networks, such as dedicated access lines, plain ordinary telephone lines, satellite links, cellular data networks, or combinations thereof. In embodiments where the network 150 comprises the Internet, data communications may take place over the network 150 via an Internet communication protocol.

Described herein are methods and systems for making recommendations of plants for advancement that include using the ensemble of the at least two or more established, i.e. trained, machine learning models, a trained deep learning model, or an ensemble of machine learning and deep learning models. The at least two or more machine learning models may be established by using, as input for training, data representations of data, representations of genotypes, phenotypes, mean locus effects (MLE) data, breeder's field notes, environmental data, predicted genetic value, pedigree information, co-ancestry information, or combinations thereof. In some examples, one or more training datasets may be selected as input for the at least two or machine learning models based on the user, environmental conditions, geographic regions, candidate genotypes, candidate phenotypes, genetic values obtained from MLE, and/or additional considerations, or combinations thereof.

While the data may be confined to one particular year of interest if desired, in some examples, the data in the training dataset is from advancement decisions for plants across multiple years, e.g. from at least 2, 3, 4, 5, 6, 7, 8, 9, 10, 11, 12, 13, 14, 15, 16, 17, 18, 19, 20, or more years.

Any ensemble of at least two or more machine learning and/or deep learning models may be trained to learn breeding strategies for one or more user's data.

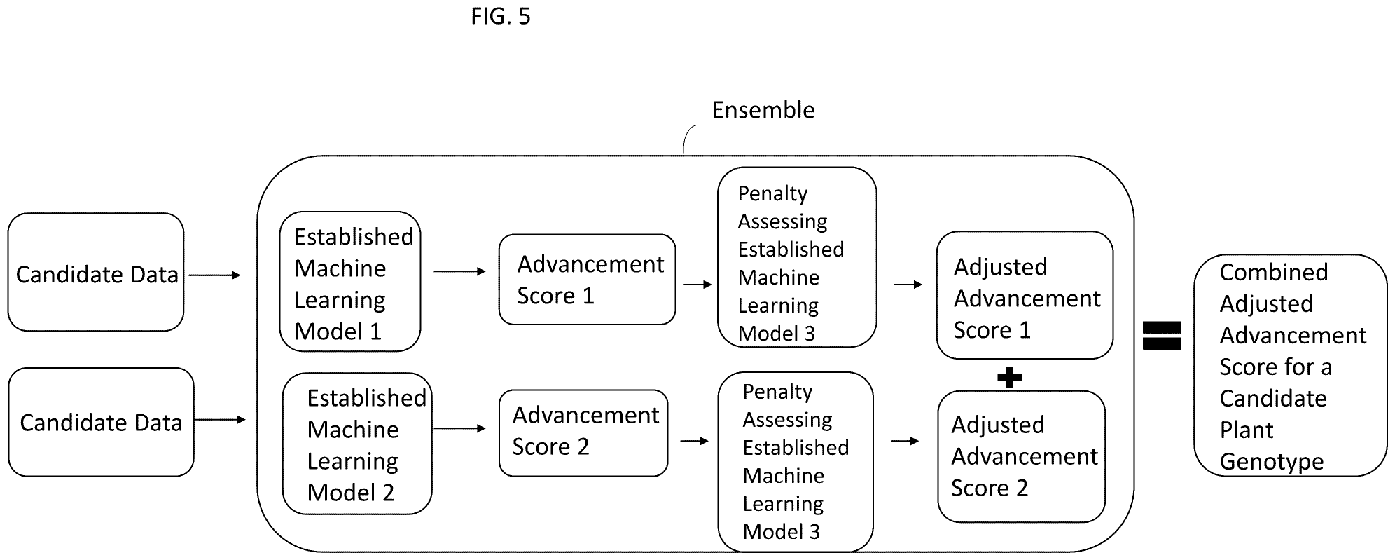

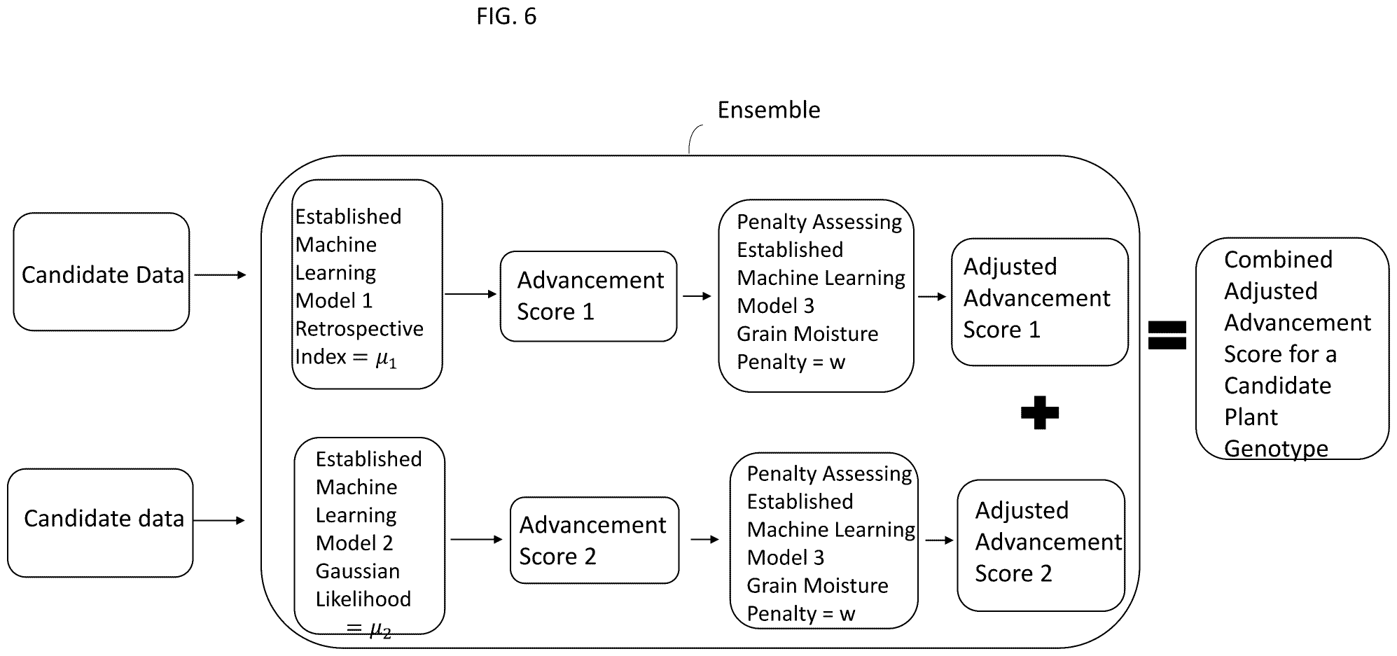

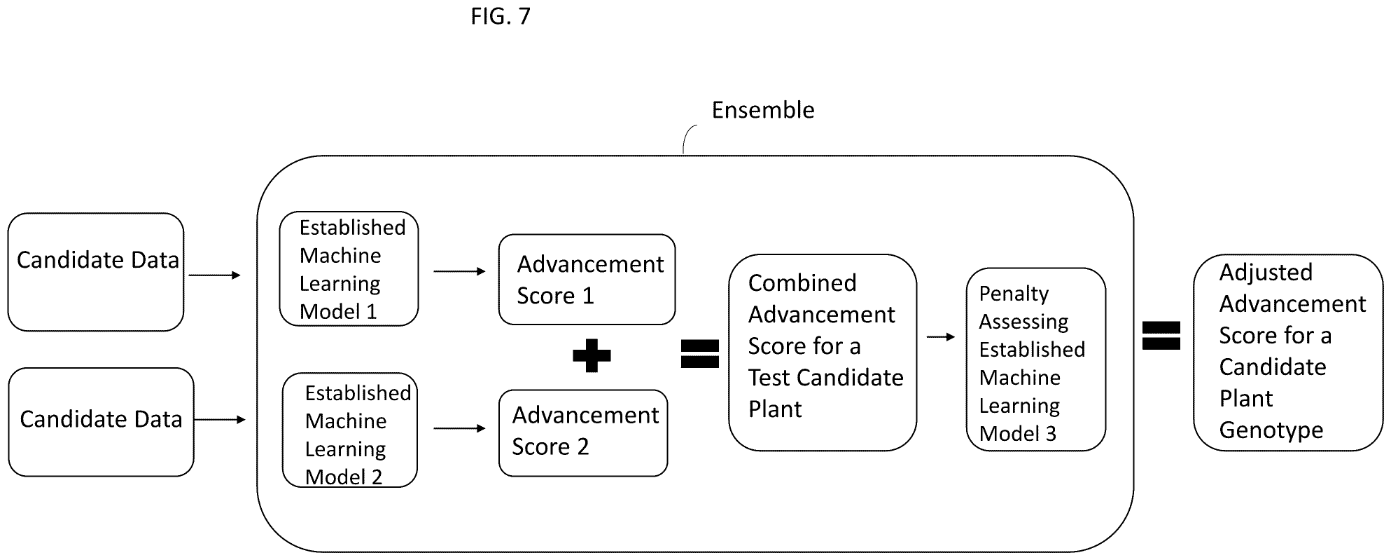

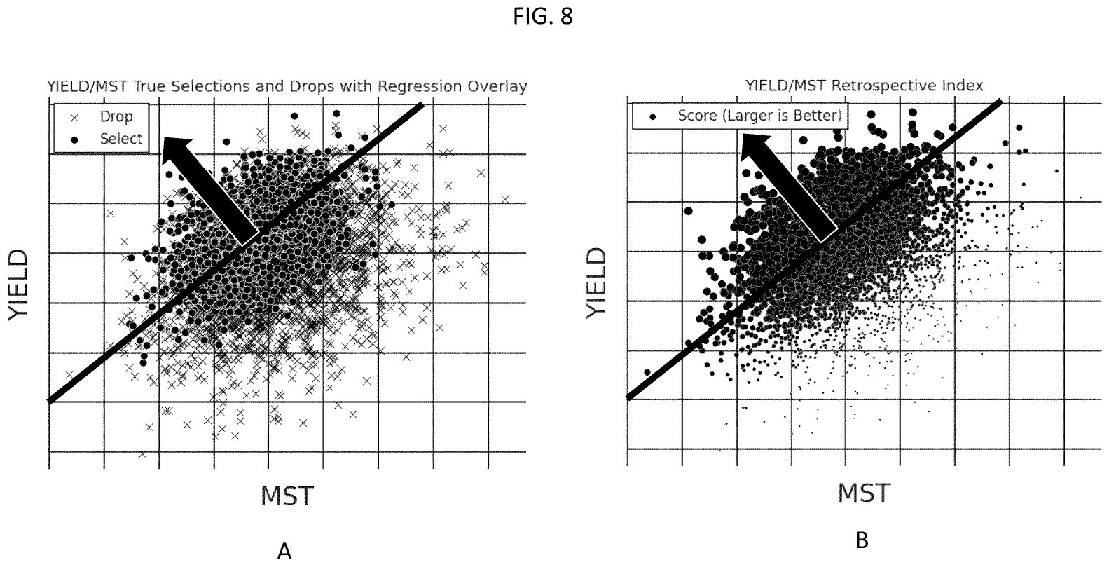

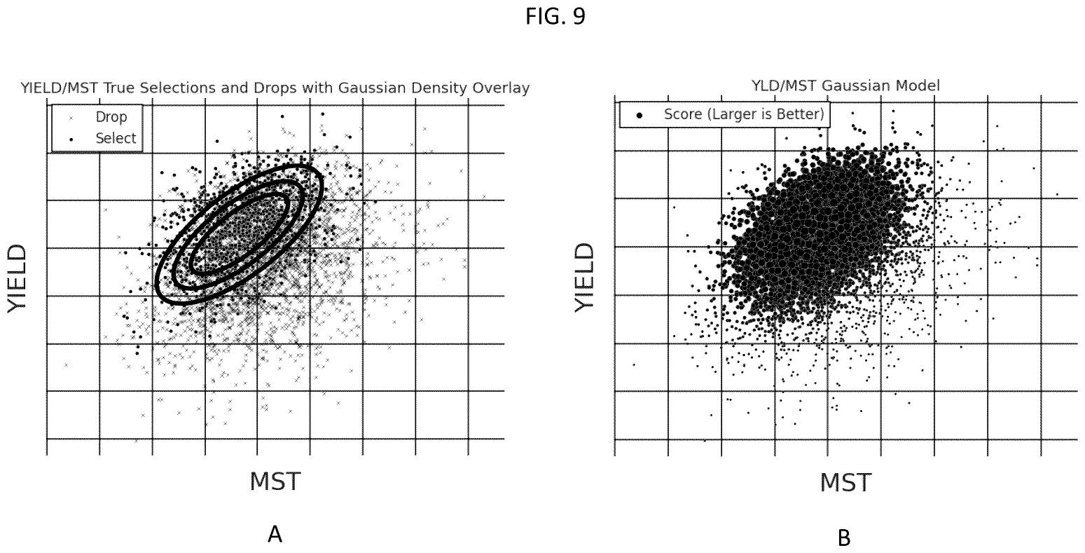

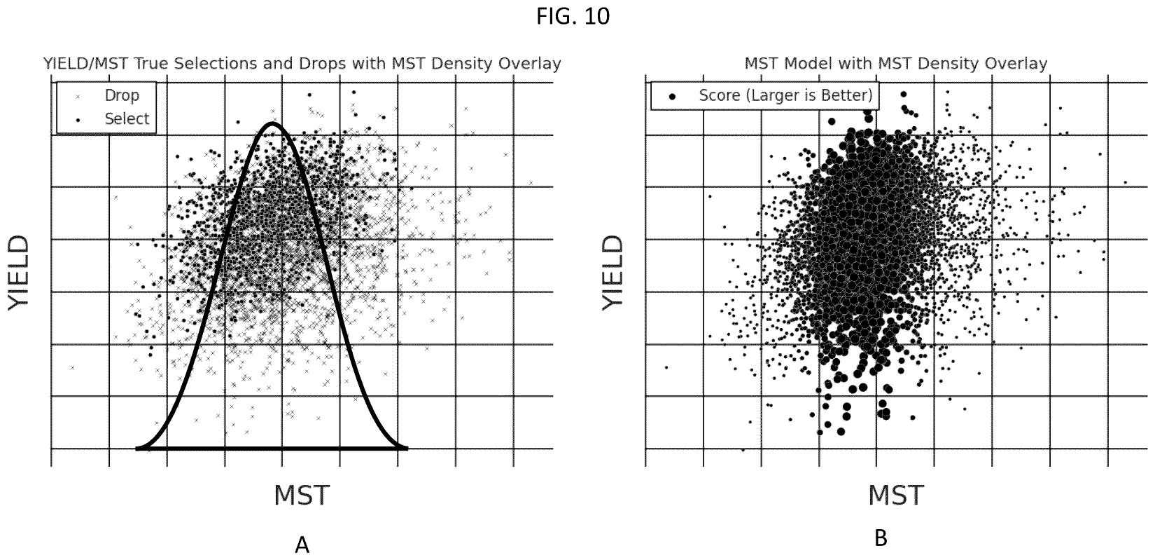

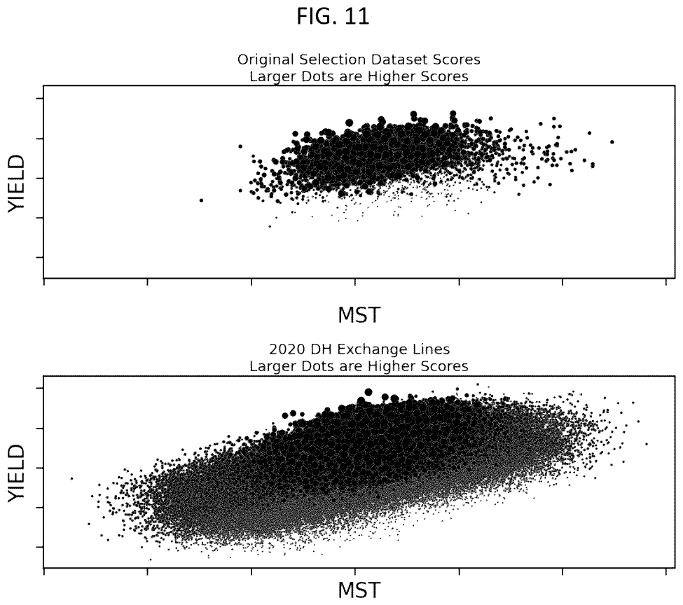

In some embodiments, the ensemble includes at least one penalty assessing machine learning model, which may be in addition to one of the at least two machine learning models. In some embodiments, the penalty-assessing machine learning model may be in an ensemble with a deep learning model, or the penalty-assessing model may be a deep learning model. As shown in Example 5, the use of the penalty assessing machine learning model may be particularly useful in those situations where it is desirable for the candidate plant genotype to meet at least one criteria, for example, a certain threshhold. As example, grain moisture may be used as a proxy for determining a maturity appropriate for a user's target market and the user may desire to consider for advancement only plants meeting that criteria. Exemplary criteria may include but are not limited to meeting a specific threshhold or range for grain moisture, ear height, plant height, yield gain, root lodging, stalk lodging, brittle snap, ear height, disease resistance, drought tolerance, diversity of genetics, and/or coancestry. In some examples, a penalty assessing machine learning model is used to provide a penalty score or penalty weight to modify the advancement score, alone or combined, generated from the at least two machine learning models, see, for example, FIGS. 5-7.

A penalty score may be applied to the individual advancement scores or combined advancement score, yielding a final combined advancement score. As shown in Example 6, a penalty score was assessed when the candidate genotypes had a high average coancestry with all other selected genotypes, and a multiplier of 0.1 was assigned to balance this penalty against performance selection.

As such, the candidates meeting or exceeding the user's threshhold for a criterion will yield a higher overall combined advancement score than those candidates that do not. In this way, the user will receive recommendations appropriate for his/her target market.

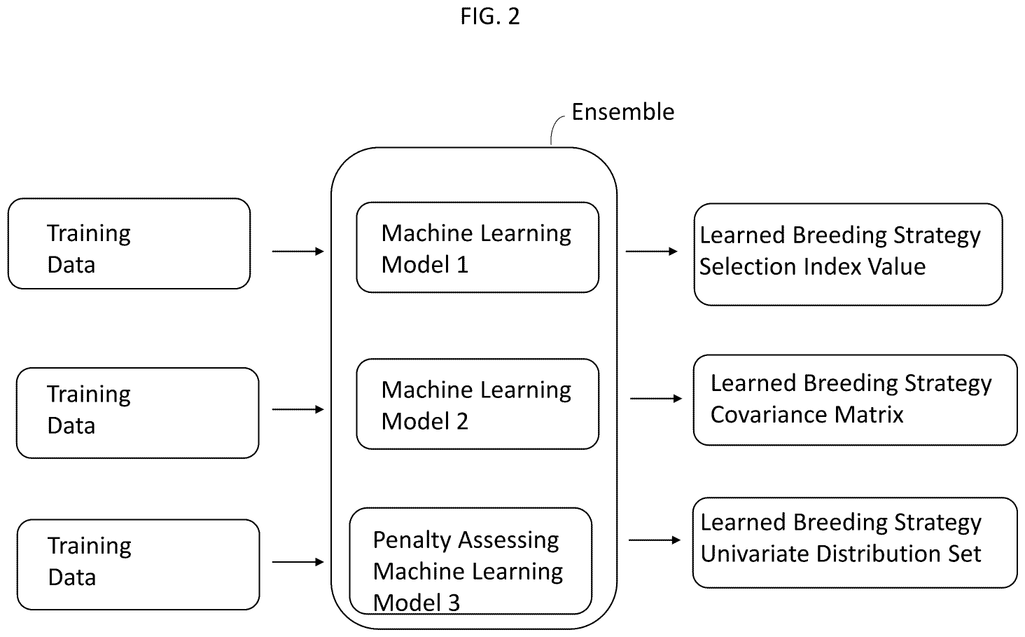

The machine learning models, including any penalty assessing machine learning models, may be trained to learn breeding strategies for one or more user's data. Referring now to FIG. 2, FIG. 2 is a schematic diagram illustrating one embodiment of using data from one or more training datasets to train the ensemble of different machine learning models to learn the breeding strategies of one or more users, for example, to result in a learned breeding strategy selection index value, covariance matrix, or univariate distribution set. The learned breeding strategies, including any resulting MLEs, scaling coefficients, and model parameters, may be written and stored so it may used and utilized with new candidate plant genotypes.

Any suitable machine learning models may be used in the methods and systems described herein. Types of models include without limitation statistical models, such as probability models, regression models, and those involving deep learning, such as supervised and unsupervised models, or combinations thereof. In some aspects, the machine learning model is a classification model, a regression model, a clustering model, a dimensionality reduction model, retrospective index model, a distribution model, for example, a multivariate or univariate Gaussian distribution model, or a deep learning model. In some embodiments, the deep learning model may be part of an ensemble model. In other embodiments, the deep learning model may be used alone or structured to provide an ensembled prediction of multiple deep learning submodels. In some embodiments, the deep learning model is a supervised learning model or a self-supervised model. In some embodiments, the deep learning model implements self-attention The supervised learning model may be a classification or regression model. In some embodiments, the deep learning model is a supervised learning model. The machine learning models include support vector machines, artificial neural networks, generalized linear regressions, generalized additive models, decision trees, ensembles of decision trees such as gradient boosted trees or random forest, splines, Gaussian processes, K-nearest neighbor predictors, or deep neural networks.

In some examples, the methods and systems described herein for making recommendations of plant genotypes for advancement include inputting the data from candidate plants genotypes into the ensemble of the at least two established machine learning models. The candidate plant data may include but is not limited to data representations of genotypes, phenotypes, mean locus effects (MLE) data, breeder's field notes, environmental data, predicted genetic values, pedigree information, co-ancestry information, or combinations thereof.

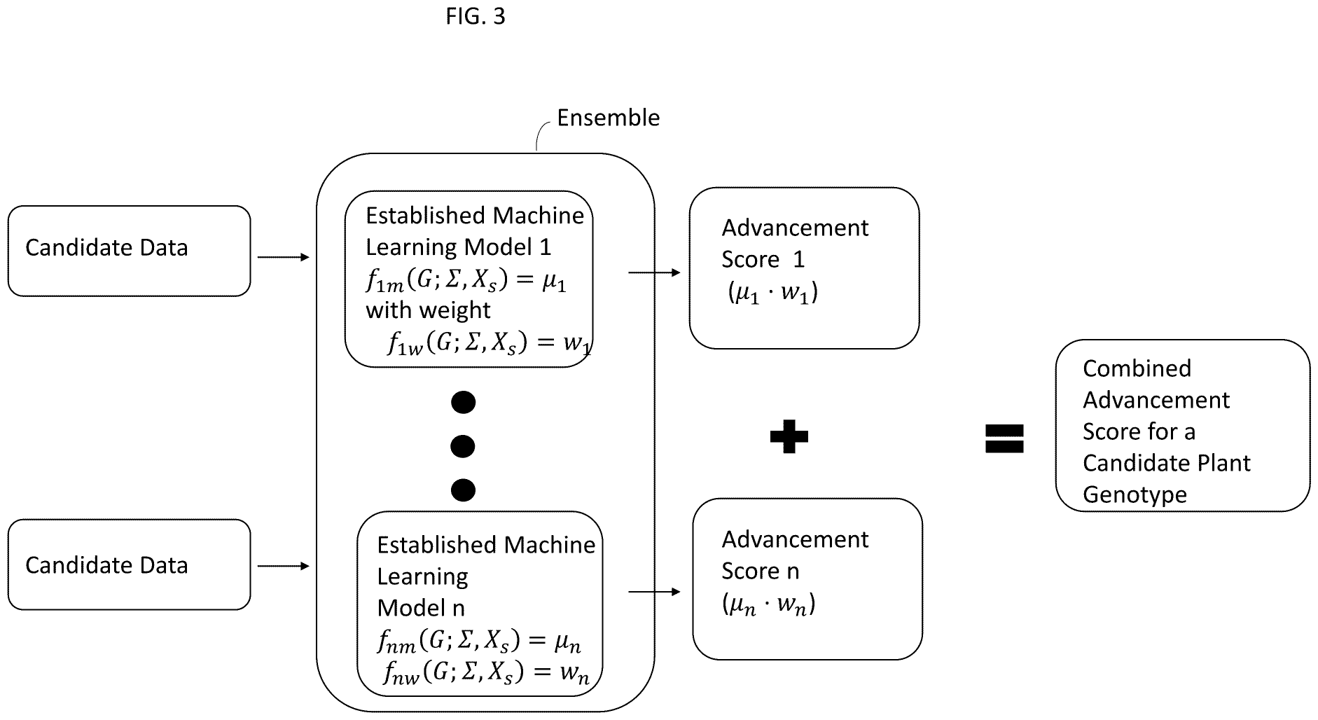

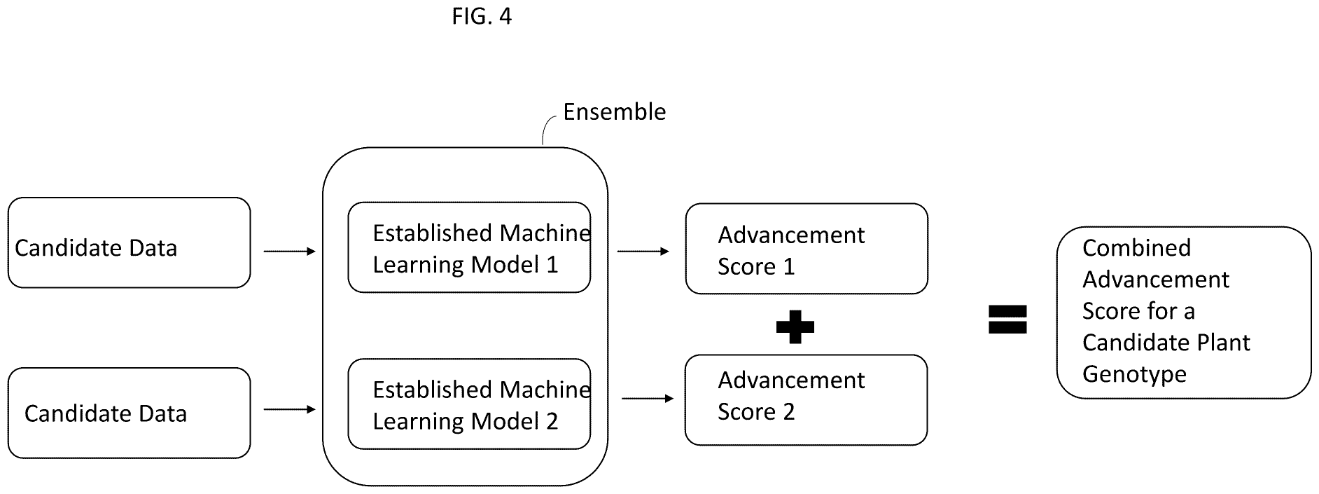

The established machine learning models may be used to generate advancement scores for each candidate plant genotype. Referring now to FIG. 3, FIG. 3 is a schematic diagram illustrating one embodiment of an ensemble with n number of different established machine learning models that each use a learned breeding strategy with candidate data from new advancement decision datasets to generate an advancement score for each of the candidates. In some aspects, each of these machine learning models, with the exception of the penalty assessing machine learning model, generates an individual advancement score for the candidate plant genotype. A combined advancement score for each candidate plant genotype may be calculated by adding the individual advancement scores from each machine learning model together.

In some examples, the processor is configured to assign a penalty to the individual or final combined advancement scores for a candidate plant genotype. A penalty score may be applied to the individual advancement scores or combined advancement score, yielding a final combined advancement score. As such, the candidates meeting or exceeding the user's threshold for a criterion will yield a higher final combined advancement score than those candidates that do not.

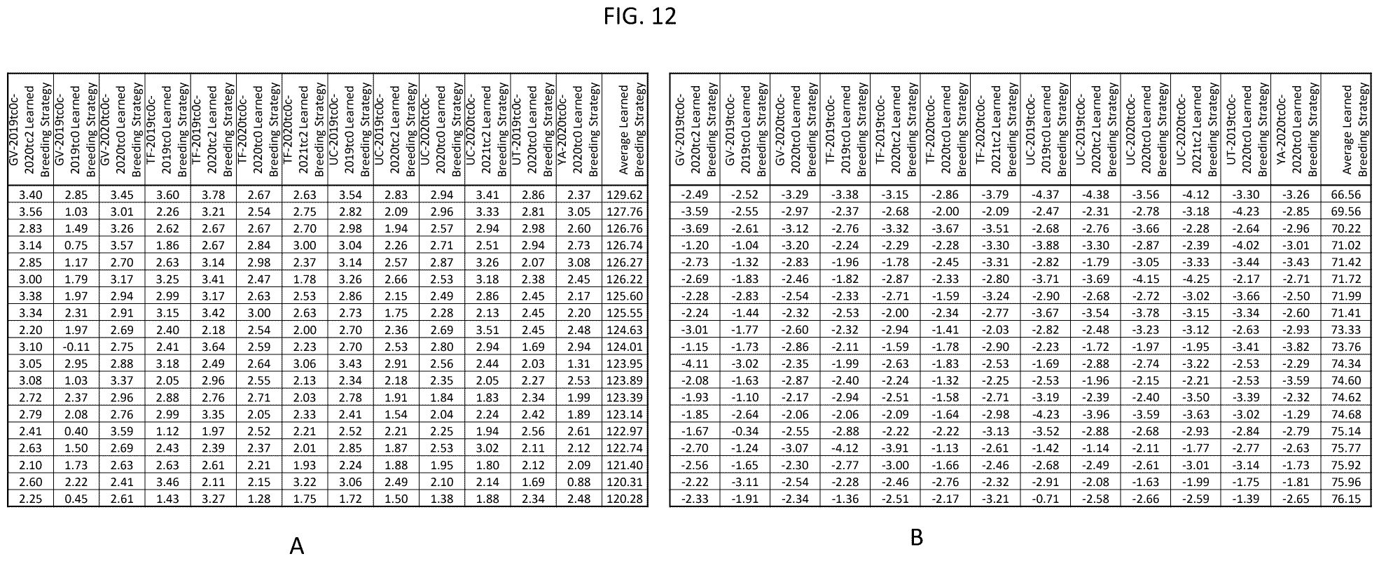

Using the systems and the methods described herein, the user will receive a quantification for each candidate plant genotype (in terms of advancement score(s)), and a collection of advancement scores for all candidates in the advancement decision datasets. The ranking, sorting, filtering, or selecting steps of the candidate plant genotypes may be performed by the computer or user or combinations thereof. The results from the ensemble, for example, the advancement scores for identified candidate plant genotypes for each learned breeding strategy, may be displayed on a user interface. One example of information that may be displayed on an interface is shown in FIG. 12.

Some embodiments of the methods may include ranking, sorting, filtering, or selecting, or combinations thereof, the candidate plant genotypes with respect to one another based on their advancement scores, e.g. individual advancement scores, combined advancement scores, or final combined advancement scores.

In some embodiments of the system, a processor is configured to rank, sort, filter, or select the candidate plant genotypes with respect to one another based on their advancement scores, e.g. individual advancement scores, combined advancement scores, or final combined advancement scores.

For example, in one embodiment, the rank may be determined by comparing the final combined advancement score for a candidate plant genotype compared to final combined advancement scores associated with other candidate plant genotypes. The candidate plant genotypes may be ranked in a numerically increasing or decreasing order, for example, using sorting. The ranking and sorting steps may be performed by the user or processer or combinations of both.

Some embodiments of the methods may include ranking, sorting, filtering, or selecting, or combinations thereof, the candidate plant genotypes with respect to one another based on whether a candidate plant genotype satisfies a given threshold value, for example, the candidate plant genotype has an individual advancement score, combined advancement score, or final advancement score that meets or exceeds the user's threshold value.

In some embodiments of the system, a processor is configured to rank, sort, filter, or select the candidate plant genotypes with respect to one another based on whether a candidate plant genotype satisfies a given threshold value, for example, the candidate plant genotype has an individual advancement score, combined advancement score, or final advancement score that meets or exceeds the user's threshold value.

In some examples, the methods and systems may optionally include filtering, by the user or processer, the candidate plant genotypes to remove from view those genotypes that do not meet the desired threshold value, individual advancement score, combined advancement score, or final advancement score, or fall within a desired percentile.

In some examples, the results are ranked based on the final combined advancement score or filtered based on a particular threshold, including with or without penalties applied. The results may be refined based on a user's preference, for example, restricting the results to a certain number of plant genotypes having the highest final advancement scores, having final advancement scores in a given percentile, for example, the top 10, 20, 30, 40, or 50 percentile of the results and/or bottom 10, 20, 30, 40, or 50 percentile of the results, or to those plants having or exceeding a threshold value or being within a certain percentile, e.g. top 10%. In some embodiments, at least 10%, 20%, 30%, 40%, 50%, 60%, 70%, 80%, 90%, 95% or more of the candidate plant genotypes having an advancement score in the top 10 percentile, top 15 percentile, top 20 percentile, top 25 percentile, or top 30 percentile of those candidate plant genotypes under consideration are advanced. In some embodiments, at least 10%, 20%, 30%, 40%, 50%, 60%, 70%, 80%, 90%, 95% or more of the candidate plant genotypes having an advancement score in the bottom 10 percentile, bottom 15 percentile, bottom 20 percentile, bottom 25 percentile, bottom 30 percentile, bottom 35 percentile, bottom 40 percentile, or bottom 50 percentile of those candidate plant genotypes under consideration are dropped from advancement or advancing through the pipeline. In some examples, the methods and systems include displaying the selected percentile of candidate plant genotypes.

In some examples, the results are ranked based on the final combined advancement score or filtered based on a particular threshold, including those with penalties applied. The results may be refined based on a user's preference, for example, restricting the results to a subset of those plants having final advancement scores in a given percentile, for example, the top ten percentile and/or bottom ten percentile. In some examples, the system and methods remove from consideration the candidate plant genotypes that do not meet the set threshold or percentile, so they are not displayed. In some examples, the system and methods select those candidate plant genotypes, or a subset of those candidate plant genotypes, meeting the specified desired threshold or percentile for display. In some examples, the systems and methods include providing different ranked lists based on different learned breeding strategies, penalties, thresholds, percentile, and candidate plant genotypes.

The user may be presented with recommendations for his/her consideration of certain plant genotypes for advancement or for non-advancement consideration based on analysis of previous advancement selection decisions, and allows for the efficient evaluation of a new advancement decision using a previous breeding strategy without having to recreate it. A final combined advancement score may be used to facilitate advancement decisions of candidates, enabling the selection and creation of improved breeding lines, progeny such as populations, and a robust genetic gain pipeline in a breeding program. For example, in some embodiments, the systems and methods include selecting one or more candidate plant genotypes based on its advancement score.

In some embodiments, the learned breeding strategies for a user/group of breeders may be stored locally or remotely stored and optionally stored as custom preferences for the user. In one example, the systems or methods may receive one or more plant candidate datasets uploaded from one or more users. In another example, the system performs the selection of the candidate plant genotypes. In some examples, the selection is based on user, e.g., operator or end-user, input.

In some embodiments, advancement scores may be averaged for one or more breeders within a certain geographic region (such as an evaluation zone (EZ)) or for a particular target market or breeding target environment. As used herein, a breeding target environment includes but is not limited to one or more particular genographic zones, evaluation zones, breeding programs, or commercial market segments, including but not limited to drought, high density planting, or heavy disease stress. Accordingly, in some examples, the ranking of the candidate plant genotypes is based on the average advancement scores from one or more breeders and the selecting of the one or more candidate plant genotypes is based on the ranking of the candidate plant genotypes.

In some examples, candidate plant genotypes that are of high interest to multiple breeders/programs may be identified using the systems and methods disclosed herein and fast-tracked for accelerated activities such as recombination and population creation from crosses of selected parents prior to field testing and coding, if desired.

In some embodiments, advancement scores may be used to identify new germplasm to introduce into a breeding program, for example, drought-tolerant doubled haploids from an alternative breeding target environment, to meet a future need. In some embodiments, advancement scores may be used to cull or remove candidates from a breeding program or eliminate them at an earlier stage, for example, as double haploids, if they are a poor fit for the future breeding pipeline. In some embodiments, candidate plant genotypes may be selected based on their advancement scores. Plants of the selected candidate plant genotypes or parts thereof may be grown in a field, greenhouse, or laboratory setting.

In some embodiments, a microspore, an embryo, or seed from a selected candidate plant may be used to generate a plant, including but not limited to a doubled haploid plant, inbred, hybrid plant, population, or derivative or offspring thereof.

In some examples, the chromosomes may be doubled at the microspore stage, at the embryo stage, at the mature seed stage, or anytime between pollination of the plant and before the germination of the haploid seed. At the microspore stage, the microspores may be treated with a diploidization agent in order to obtain a doubled haploid embryo, which may then be grown into a doubled haploid plant. For instance, microspores may be placed in contact with a chromosome doubling agent such as colchicine or herbicides like amiprophos methyl, oryzalin, and pronamide. A chromosome doubling agent may also be applied to multicellular clusters, pro-embryoids, or somatic embryos (any actively dividing cell). A microspore selected using the methods provided herein may also be used to fertilize a female gametic cell.

In the case of pollen grains, if selected, a pollen grain may be used for pollination, enabling the fertilization of a female gamete and the development of a seed that may be grown into a plant.

In some embodiments, the selected candidate plant may be crossed with a maternal inducer line to produce seeds with haploid embryos. In some embodiments, the selected candidate plant may be crossed with itself to create an improved inbred population having desirable (improved) characteristics. The candidate plant may also be self-crossed (“selfed”) to create a true breeding line with the same genotype.

In some embodiments, the selected candidate plant may be crossed with another candidate plant or other breeding plant to create an improved offspring (hybrid) with desirable or improved characteristics, improved hybrid vigor, or combinations thereof. In some embodiments, the selected candidate plant may be used in crosses to generate a population of progeny. The selected candidate plant may also be outcrossed, e.g., to a plant or line not present in its genealogy. The selected candidate plant may be introduced into the breeding program de novo.

In some embodiments, the selected candidate plant may be used in recurrent selection, bulk selection, mass selection, backcrossing, pedigree breeding, open pollination breeding, restriction fragment length polymorphism enhanced selection, genetic marker enhanced selection, double haploids, transformation, and/or gene editing. As an example, the selected candidate plant or part thereof may be targeted for gene editing using CRISPR/CAS, Zn Fingers, meganucleases, TALENs, or any combination thereof, to either generate a favorable genetic composition in a specific region of the genome or to introduce characteristics or traits that facilitate further growth and development.

### Embodiments

The present disclosure is further illustrated in the following embodiments. It should be understood that these embodiments are given by way of illustration only.

Embodiment 1. A method for use in breeding comprising:
(a) receiving, through one or more computing devices, at least one training data set comprising data from a breeder's selections of plants for advancement;
(b) inputting the data from the training data set into an ensemble of at least two machine learning models;
(c) training the ensemble of the at least two machine learning models to learn the likelihood of advancement of a plant genotype from the training data set;
(d) inputting candidate data comprising data from candidate plant genotypes being considered for advancement into the ensemble of the at least two trained machine learning models; and
(e) generating by the ensemble an advancement score for each candidate plant genotype.

Embodiment 2. A computer-implemented training method comprising:
(a) receiving by a deep learning model implementing self-attention in a computing device one or more plant genotype representations and one or more representations of breeding target environments associated with the training plant genotype representations;
(b) simulatenously learning by the deep learning model an association among genotypes and between plant genotypes and breeding target environments to produce a prediction whether plant genotypes are associated with one another and with a given breeding target environment;
(c) evaluating a loss function of the predicted associations among the plant genotypes and predicted associations among the plant genotypes and breeding target environments with respect to their true grouping values;
(d) adjusting the weights of the embeddings, and/or the self-attention model and/or the predictive output layer of the tokens to reduce the evaluated loss; and
(e) reiterating steps (a)-(d).

Embodiment 3. A method for use in breeding comprising:
(a) receiving input data data comprising data from candidate plant genotypes being considered for advancement through a computing device;
(b) inputting candidate data comprising data from candidate plant genotypes being considered for advancement into the ensemble of the at least two trained machine learning models, wherein the at least two trained machine learning models have been trained to learn the likelihood of advancement of a plant; and
(c) generating by the ensemble an advancement score for each candidate plant genotype.

Embodiment 4. A computer-implemented fine-tuning method comprising:
(a) receiving by the pre-trained deep learning model implementing self-attention in a computing device one or more plant genotype representations and one or more representations of breeding target environments associated with the plant genotype representations, wherein the plant genotype representations comprise token embeddings and plurality breeding target environment representations are tokens to produced a predicted advancement score; and
(b) evaluating the loss function of the predicted advancement score for each plant genotypes with respect to their true advancement values;
(c) adjusting one or more of the weights of the token embeddings, and/or the self-attention model, and/or the predictive output layer of the tokens to reduce the evaluated loss; and
(d) reiterating steps (a)-(c).

Embodiment 5. A computer-implemented prediction method comprising:
(a) inputting into the trained deep learning model a plurality of candidate plant genotypes that a breeding target environment is considering along with the breeding target environment token that is considering the plurality of candidate plant genotypes to produce a predictive advancement score for each candidate plant genotype.

The embodiment of 5 may include prior to step (a):
(1) receiving by a deep learning model implementing self-attention in a computing device one or more plant genotype representations and one or more representations of breeding target environments associated with the training plant genotype representations;
(2) simultaneously learning by the deep learning model an association among genotypes and between plant genotypes and breeding target environments to produce a prediction whether plant genotypes are associated with one another and with a given breeding to produce predicted associations of a given genotype and predicted associations of the breeding target environment with all the plant genotypes;
(3) evaluating the loss function of the predicted associations among the plant genotypes and predicted associations among the plant genotypes and breeding target environments with respect to their true grouping values;
(4) adjusting the weights, e.g. of the token embeddings, and/or the self-attention model, and/or the predictive output layer of the tokens to reduce the error based on the loss-function evaluation;
(5) reiterating steps (1)-(4);
(6) receiving by the trained deep learning model implementing self-attention in a computing device one or more candidate plant genotype representations and one or more representations of breeding target environments associated with the candidate plant genotype representations, wherein the plant genotype representations comprise tokens and plurality breeding target environment representations are tokens to produce a predicted an advancement score for each plant genotype.

Embodiment 6. A method for creating a doubled haploid plant, the method comprising:
(a) receiving, through one or more computing devices, at least one training data set comprising data from a breeder's selections of plants for advancement;
(b) inputting the data from the training data set into an ensemble of at least two machine learning models;
(c) training the ensemble of the at least two machine learning models to learn the likelihood of advancement of a plant from the training data set;
(d) generating by the ensemble an advancement score for each candidate plant genotype;
(e) selecting one or more candidate plant genotypes based on its advancement score;
(f) growing one or more of the selected candidates, e.g. one or more plants of the selected candidate plant genotypes or parts thereof;
(g) obtaining a tetrad microspore from the one or more selected candidates;
(h) contacting the tetrad microspore with a chromosome doubling agent to produce a doubled haploid embryo; and
(i) generating a doubled haploid plant from the doubled haploid embryo.

Embodiment 7. A method for creating an improved plant or population of plants, the method comprising:
(a) receiving, through one or more computing devices, at least one training data set comprising data from a breeder's selections of plants for advancement;
(b) inputting the data from the training data set into an ensemble of at least two machine learning models;
(c) training the ensemble of the at least two machine learning models to learn the likelihood of advancement of a plant from the training data set;
(d) inputting candidate data comprising data from candidate plant genotypes being considered for advancement into the ensemble of the at least two trained machine learning models;
(e) generating by the ensemble an advancement score for each candidate plant genotype;
(f) selecting one or more candidate plant genotypes based on its advancement score;
(g) growing one or more of the selected candidates, e.g. one or more plants of the selected candidate plant genotypes or parts thereof; and
(h) crossing one or more of the selected candidates with (1) a maternal inducer line to produce seeds with haploid embryos, (2) itself to create an improved inbred population having desirable (improved) characteristics, or
(3) another candidate or breeding plant to create an improved offspring (hybrid) with desirable (improved) characteristics, improved hybrid vigor, or combinations thereof.

Embodiment 8. A method for creating a doubled haploid plant comprising:
(a) receiving by a deep learning model implementing self-attention in a computing device one or more plant genotype representations and one or more representations of breeding target environments associated with the training plant genotype representations;
(b) simulatenously learning by the deep learning model an association among genotypes and between plant genotypes and breeding target environments to produce a prediction whether plant genotypes are associated with one another and with a given breeding to produce predicted associations of a given genotype and predicted associations of the breeding target environment with all the plant genotypes;
(c) evaluating the loss function of the predicted associations among the plant genotypes and predicted associations among the plant genotypes and breeding target environments with respect to their true grouping values;
(d) adjusting the weights, e.g. of the token embeddings, and/or the self-attention model, and/or the predictive output layer of the tokens to reduce the error based on the loss-function evaluation;
(e) reiterating steps (a)-(d);
(f) receiving by the pre-trained deep learning model implementing self-attention in a computing device one or more plant genotype representations and one or more representations of breeding target environments associated with the plant genotype representations, wherein the plant genotype representations comprise tokens and plurality breeding target environment representations are tokens to produce a predicted advancement score;
(g) evaluating the loss function of the predicted advancement score for each plant genotypes with respect to their true advancement values;
(h) reiterating steps (f)-(g);
(i) inputting into the trained deep learning model a plurality of plant genotypes that a breeding target environment is considering along with the breeding target environment token that is considering the plurality of genotypes to produce a predictive advancement score for each plant genotype;
(j) selecting one or more candidate plant genotypes based on its advancement score;
(k) growing one or more of the selected candidates, e.g. one or more plants of the selected candidate plant genotypes or parts thereof;
(l) obtaining a tetrad microspore from the one or more selected candidates;
(m) contacting the tetrad microspore with a chromosome doubling agent to produce a doubled haploid embryo; and
(n) generating a doubled haploid plant from the doubled haploid embryo.

Embodiment 9. A method for creating an improved plant or population of plants, the method comprising:
(a) receiving by a deep learning model implementing self-attention in a computing device one or more plant genotype representations and one or more representations of breeding target environments associated with the training plant genotype representations;
(b) simulatenously learning by the deep learning model an association among genotypes and between plant genotypes and breeding target environments to produce a prediction whether plant genotypes are associated with one another and with a given breeding to produce predicted associations of a given genotype and predicted associations of the breeding target environment with all the plant genotypes;
(c) evaluating the loss function of the predicted associations among the plant genotypes and predicted associations among the plant genotypes and breeding target environments with respect to their true grouping values;
(d) adjusting the weights, e.g. of the token embeddings, and/or the self-attention model, and/or the predictive output layer of the tokens to reduce the error based on the loss-function evaluation;
(e) reiterating steps (a)-(d);
(f) receiving by the pre-trained deep learning model implementing self-attention in a computing device one or more plant genotype representations and one or more representations of breeding target environments associated with the plant genotype representations, wherein the plant genotype representations comprise tokens and plurality breeding target environment representations are tokens to produced a predicted advancement score;
(g) evaluating the loss function of the predicted advancement score for each plant genotypes with respect to their true advancement values;
(h) reiterating steps (f)-(g);
(i) inputting into the trained deep learning model a plurality of candidiate plant genotypes that a breeding target environment is considering along with the breeding target environment token that is considering the plurality of genotypes to produce a predictive advancement score for each plant genotype;
(j) selecting one or more candidate plant genotypes based on its advancement score;
(k) growing one or more of the selected candidates, e.g. one or more plants of the selected candidate plant genotypes or parts thereof; and (l) crossing one or more of the selected candidates with (1) a maternal inducer line to produce seeds with haploid embryos, (2) itself to create an improved inbred population having desirable (improved) characteristics, or
(3) another candidate or breeding plant to create an improved offspring (hybrid) with desirable (improved) characteristics, improved hybrid vigor, or combinations thereof.

Embodiment 10. The method of any of the embodiments of embodiments 1, 3, 6, or 7, the method further comprising: generating by the ensemble an advancement score for each candidate plant genotype for a particular environment or region.

Embodiment 11. The method of any of the embodiments of embodiments 1, 3, 6, or 7, the method further comprising: generating by the ensemble an advancement score for each candidate plant genotype for a particular environment or region.

Embodiment 12. The method of of any of the embodiments of embodiments 1, 3, 6, or 7, the method further comprising: generating by the ensemble an advancement score for each candidate plant genotype for a particular characteristic/trait.

Embodiment 13. The method of of any of the embodiments of embodiments 1, 3, 6, or 7, the method further comprising: generating by the ensemble average advancement scores from two or more breeders for each candidate plant genotype.

Embodiment 14. The method of any of the embodiments of embodiments 1, 3, 6, or 7, the method further comprising: generating by the ensemble average advancement scores from two or more breeders for each candidate plant genotype for a particular environment or region.

Embodiment 15. The method of any of the embodiments of embodiments 1, 3, 6, or 7, the method further comprising: generating by the ensemble average advancement scores from two or more breeders for each candidate plant genotype for a particular characteristic/trait for a particular environment or region.

Embodiment 16. The method of any of the embodiments of embodiments 2, 4, 5, 8, or 9, the method further comprising: generating by the deep learning model an advancement score for each candidate plant genotype for a particular environment or region.

Embodiment 17. The method of any of the embodiments of embodiments 2, 4, 5, 8, or 9, the method further comprising: generating by the deep learning model an advancement score for each candidate plant genotype for a particular environment or region.

Embodiment 18. The method of any of the embodiments of embodiments 2, 4, 5, 8, or 9, the method further comprising: generating by the deep learning model an advancement score for each candidate plant genotype for a particular characteristic/trait.

Embodiment 19. The method of any of the embodiments of embodiments 2, 4, 5, 8, or 9, the method further comprising: generating by the deep learning model average advancement scores from two or more breeders for each candidate plant genotype.

Embodiment 20. The method of any of the embodiments of embodiments 2, 4, 5, 8, or 9, the method further comprising: generating by the deep learning model average advancement scores from two or more breeders for each candidate plant genotype for a particular environment or region.

Embodiment 21. The method of any of the embodiments of embodiments 2, 4, 5, 8, or 9, the method further comprising: generating by the deep learning model average advancement scores from two or more breeders for each candidate plant genotype for a particular characteristic/trait for a particular environment or region.

Embodiment 22. The method of any of the preceeding embodiments, the method comprising presenting the advancement score for each candidate plant genotype on a user interface or display.

Embodiment 23. The method of any of the embodiments of embodiments 1-22, the method comprising selecting one or more candidate plant genotypes based on its advancement score.

Embodiment 24. The method of any of the embodiments of embodiments 1-23, wherein the plant genotype is for a monocot or dicot plant.

Embodiment 25. The method of any of the embodiments of embodiments 1-24, wherein the plant genotype is for a soybean, maize, sorghum, cotton, canola, sunflower, rice, wheat, sugarcane, alfalfa tobacco, barley, cassava, peanuts, millet, oil palm, potatoes, rye, or sugar beet plant.

Embodiment 26. The method of any of the embodiments of embodiments 2, 4, 5, 8, or 9, wherein the representations of plant genotypes and representations of the breeding target environments are tokens with vector embeddings.

Embodiment 27. The method of any of the embodiments of embodiments 2, 4, 5, 8, or 9, wherein an output of the deep learning model is a binary output for each plant genotype.

Embodiment 28. The method of any of the embodiments of embodiments 2, 4, 5, 8, or 9, wherein the method comprises transforming the SNPs of the plant genotypes or candidate plant genotypes into plant genotype representations prior to step (a).

Embodiment 29. The method of any of the embodiments of embodiments 2, 4, 5, 8, or 9, wherein the method comprises transforming genotypic information of the plant genotypes or candidate plant genotypes into plant genotype representations prior to step (a).

Embodiment 30. The method of any of the embodiments of embodiments 1-29, wherein the method comprises determining individual advancement scores for each of the candidate plant genotypes.

Embodiment 31. The method of any of the embodiments of embodiments 1, 3, 6, or 7, wherein the method comprises determining a combined advancement score for each of the candidate plant genotypes based on a combination of the individual advancement scores from each of the machine learning models.

Embodiment 32. The method any of the embodiments of embodiments 1, 3, 6, or 7, wherein the method of determining the advancement score or a final (overall) advancement score for each of the candidate plant genotypes includes assessing a penalty.

Embodiment 33. The method of any of the embodiments of embodiments 1-32, the method further comprising averaging the advancement scores for two or more breeders.

Embodiment 34. The method of any of the embodiments of embodiments 1-33, the method further comprising averaging the advancement scores for two or more breeders within a certain geographic region or for a particular target market.

Embodiment 35. The method of any of the embodiments of embodiments 1-34, wherein the method comprises selecting a subset of candidate plant genotypes from a plurality of candidate plant genotypes based on the advancement scores of the candidate plant genotypes meeting a given threshold value for an advancement score.

Embodiment 36. The method of any of the embodiments of embodiments 1-34, wherein the method comprises selecting a subset of candidate plant genotypes from a plurality of candidate plant genotypes based on the advancement scores of the candidate plant genotypes being within a given percentile of the candidate plant genotypes.

Embodiment 37. The method of any of the embodiments of embodiments 1-34, wherein the method comprises selecting a subset of candidate plant genotypes from a plurality of candidate plant genotypes based on a certain number of plant genotypes having the highest or lowest advancement scores.

Embodiment 38. The method of any of the embodiments of embodiments 1-34, wherein the method comprises determining a ranking of the candidate plant genotypes based on the advancement score for each candidate plant genotype.

Embodiment 39. The method of any of the embodiments of embodiments 1-34, the method further comprising:
determining a ranking of the candidate plant genotypes based on the average advancement scores from two or more breeders, wherein selecting the one or more candidate plant genotypes is based on the ranking of the candidate plant genotypes.

Embodiment 40. The method of any of the embodiments of embodiments 1-34, the method further comprising:
determining a ranking of the candidate plant genotypes based on the average advancement scores from two or more breeders, wherein selecting the one or more candidate plant genotypes is based on the ranking of the candidate plant genotypes having the highest advancement scores for a particular region.

Embodiment 41. The method of embodiment 6 or 8, the method further comprising treating the haploid embryos with a doubling agent to make a doubled haploid embryo.

Embodiment 42. The method of embodiment 41, further comprising generating a doubled haploid plant from the doubled haploid embryo.

Embodiment 43. The method of embodiment 42, further comprising allowing the doubled haploid plant to self-pollinate to produce completely homozygous seeds, wherein the doubled haploid plant is an inbred plant.

Embodiment 44. A computer readable medium having stored thereon instructions to provide candidate plant genotypes recommendations, when executed by a processor (or computing device), cause the processor to perform the steps of embodiment 1.

Embodiment 45. A computer readable medium having stored thereon instructions to provide candidate plant genotypes recommendations, when executed by a processor (or computing device), cause the processor to perform the steps of embodiment 2.

Embodiment 46. A computer readable medium having stored thereon instructions to provide candidate plant genotypes recommendations, when executed by a processor (or computing device), cause the processor to perform the steps of embodiment 3.

Embodiment 47. A computer readable medium having stored thereon instructions to provide candidate plant genotypes recommendations, when executed by a processor (or computing device), cause the processor to perform the steps of embodiment 4.

Embodiment 48. A computer readable medium having stored thereon instructions to provide candidate plant genotypes recommendations, when executed by a processor (or computing device), cause the processor to perform the steps of embodiment 5.

Embodiment 49. A system comprising:
(a) one or more servers, each of the one or more server storing plant data; and
(b) a computing device communicatively coupled to the one or more servers, the computing device including:
   (1) a memory; and
   (2) one or more processors configured to perform operations comprising:
      (a) obtain at least one training data set comprising data from a breeder's selections of plants for advancement;
      (b) learn the likelihood of advancement of a candidate plant genotype from the training data set using an ensemble of at least two machine learning models;
      (c) obtain data from a plurality of candidate plant genotypes; and
      (d) generate an advancement score for each candidate plant genotype from the plurality of candidate plant genotypes.

Embodiment 50. A system comprising:
(a) one or more servers, each of the one or more server storing plant data; and
(b) a computing device communicatively coupled to the one or more servers, the computing device including:
   (1) a memory; and
   (2) one or more processors configured to perform operations comprising:
      (a) obtain one or more plant genotype representations and one or more representations of breeding target environments associated with the training plant genotype representations;
      (b) simulatenously learn an association among genotypes and between plant genotypes and breeding target environments to produce a prediction whether plant genotypes are associated with one another and with a given breeding target environment using a deep learning model implementing self-attention;
      (c) evaluate the loss function of the predicted associations among the plant genotypes and predicted associations among the plant genotypes and breeding target environments with respect to their true grouping values;
      (d) adjust the weights of the self-attention model, and/or the embedding model, and/or the predictive output layer of the tokens to reduce the evaluated loss; and
      (e) reiterate steps (a)-(d) until convergence of validation loss to a desired value.

Embodiment 51. A system comprising:
(a) one or more servers, each of the one or more server storing plant data; and
(b) a computing device communicatively coupled to the one or more servers, the computing device including:
   (1) a memory; and
   (2) one or more processors configured to perform operations comprising:
      (a) obtain data from a plurality of candidate plant genotypes; and
      (b) generate an advancement score for each candidate plant genotype from the plurality of candidate plant genotypes using an ensemble of at least two trained machine learning models.

Embodiment 52. A system comprising:
(a) one or more servers, each of the one or more server storing plant data; and
(b) a computing device communicatively coupled to the one or more servers, the computing device including:
   (1) a memory; and
   (2) one or more processors configured to perform operations comprising:
      (a) obtain one or more plant genotype representations and one or more representations of breeding target environments associated with the training plant genotype representations;
      (b) simulatenously learn an association among genotypes and between plant genotypes and breeding target environments to produce a prediction whether plant genotypes are associated with one another and with a given breeding target environment using a deep learning model implementing self-attention;
      (c) evaluate the loss function of the predicted associations among the plant genotypes and predicted associations among the plant genotypes and breeding target environments with respect to their true grouping values;
      (d) adjust the weights of the self-attention model, the embedding model, the predictive output layer of the tokens to reduce the evaluated loss; and
      (e) reiterate steps (a)-(d) until convergence of validation loss to a desired value;
      (f) obtain by the pre-trained deep learning model implementing self-attention one or more plant genotype representations and one or more representations of breeding target environments associated with the plant genotype representations; and
      (g) evaluate the loss function of the predicted advancement score for each of the plant genotypes with respect to their true advancement values.
      (h) adjust the weights of the self-attention model, the embedding model, the predictive output layer of the tokens to reduce the evaluated loss; and
         (i) reiterate steps (f)-(h) until convergence of validation loss to a desired value.

Embodiment 53. A system comprising:
(a) one or more servers, each of the one or more server storing plant data; and
(b) a computing device communicatively coupled to the one or more servers, the computing device including:
   (1) a memory; and
   (2) one or more processors configured to perform operations comprising:
      (a) obtain one or more plant genotype representations and one or more representations of breeding target environments associated with the training plant genotype representations;
      (b) simulatenously learn an association among genotypes and between plant genotypes and breeding target environments to produce a prediction whether plant genotypes are associated with one another and with a given breeding target environment using a deep learning model implementing self-attention;
      (c) evaluate the loss function of the predicted associations among the plant genotypes and predicted associations among the plant genotypes and breeding target environments with respect to their true grouping values;
      (d) adjust the weights of the self-attention model, the embedding model, the predictive output layer of the tokens to reduce the error based on the loss-function evaluation; and
      (e) reiterate steps (a)-(d) as until convergence of validation loss to a desired value;
      (f) obtain by the pre-trained deep learning model implementing self-attention one or more plant genotype representations and one or more representations of breeding target environments associated with the plant genotype representations; and
      (g) evaluate the loss function of the predicted advancement score for each of the plant genotypes with respect to their true advancement values.
      (h) adjust the weights of the self-attention model, the embedding model, the predictive output layer of the tokens to reduce the evaluated loss; and
      (i) reiterate steps (f)-(h) until convergence of validation loss to a desired value
      (j) receive as input into the trained deep learning model a plurality of candidate plant genotypes that a breeding target environment is considering along with the breeding target environment token that is considering the plurality of genotypes and generate an advancement score for each plant genotype.

Embodiment 54. A computer-implemented method for construction of a tokenization scheme for one or more breeder's notes, the method comprising:
(a) receiving, through one or more computing devices, one or more breeders' notes;
(b) using byte-pair encoding to encode each unique combination of one or more consecutive characters in the one or more breeder's notes into a token by recursively combining common pairs of tokens in the one or more breeder notes into single tokens until a target vocabulary is reached.

Embodiment 55. A computer-implemented method for creating a vocabulary for one or more breeder's notes, the method comprising:
(a) receiving, through one or more computing devices, one or more breeder's notes, wherein the breeder's notes comprise one or more word parts;
(b) encoding each word part in each of the one or more breeder's notes into a set of tokens for use with a tokenizer.

Embodiment 56. The method of any of the embodiments of embodiments 54 or 55 or 64, wherein the one or more word parts comprises one or more characters.

Embodiment 57. The method of any of the embodiments of embodiments 54 or 55 or 56 or 64, wherein the one or more word parts comprises an abbreviation or acronym of a word or a series of words, such as NLB as an abbreviation for Northern Leaf Blight.

Embodiment 58. The method of embodiment 54 or 55 or 64, wherein there are a plurality of tokens for a breeder's note.

Embodiment 59. The method of embodiment 55 or 64, wherein the tokenizers comprise byte-pair encoding, word piece, or sentence piece tokenizers.

Embodiment 60. The method of embodiment 54 or 55 or 64, wherein the two or more breeders' notes are in the same language, different language, or combinations thereof.

Embodiment 61. The embodiment of embodiment 54 or 55 or 64, wherein the one or more breeder notes comprise one or more word parts derived from speech, e.g. spoken words, or audio input.

Embodiment 62. The embodiment of embodiment 54 or 55 or 64, wherein the breeder note is converted to one or more word parts in text from speech or audio format.

Embodiment 63. The embodiment of embodiment 54 or 55 or 64, wherein the speech or audio input is a spoken word.

Embodiment 64. A computer-implemented method for generating a unified representation for a plant genotype for one or more breeder's notes, the method comprising:
(a) receiving by a tokenizer implementing a tokenization scheme for a constructed vocabulary for one or more breeder's notes, wherein the breeder's notes comprise one or more word parts;
(b) assigning each word part of the one or more breeder's notes a token;
(c) assigning each breeder its own unique token;
(d) receiving by a deep learning model implementing self-attention in a computing device one or more pairings of breeder and breeder's notes, wherein the breeders are tokenized, and the breeder's notes comprise one or more word parts that have been encoded into tokens using a constructed tokenizer vocabulary;
(e) converting by an embedding layer each token in the input to a unique token embedding corresponding to that token;
(f) pretraining, by the one or more processors, the deep learning model to learn structural language patterns in the breeder notes, relationships of notes to breeders, and how notes co-occur with one another among plant genotypes, the pretraining constituting a masked language modeling task comprising:
   (1) performing, by the one or more processors, selection of one or more tokens to be evaluated by the loss function following the output layer;
   (2) generating, by the one or more processors, replacement of one or more of the selected input token embeddings from (1) with either an alternative token embedding selected from the tokenizer vocabulary or a token embedding representing the masked state;
   (3) generating by the deep learning model a prediction of the true (observed) token foreach input token;
   (4) evaluating the loss function of the predicted tokens with respect to their true values for those tokens selected in (1);
   (5) adjusting the weights of the token embeddings, the self-attention model, and the predictive output layer of the tokens to reduce the evaluated loss;
   (6) reiterating steps (1)-(5) until convergence of validation loss to a desired value;
(g) pretraining, by the one or more processors, the deep learning model to learn associations between breeder's notes and the breeder who wrote the notes, the pretraining constituting a next sentence prediction task comprising:
   (1) receiving by the deep learning model a breeder token paired with the one or more tokens for a breeder note from that breeder, converting all tokens into token embeddings;
   (2) performing with a frequency between 0 and 1, by the one or more processors, replacement of the true breeder token embedding with the breeder token embedding for an alternative breeder
   (3) generating by the deep learning model a prediction of whether the breeder token and breeder note tokens correspond to the true (observed) values;
   (4) evaluating the loss function of the predicted associations between the breeder tokens and breeder note token with respect to its true value;
   (5) adjusting the weights of the token embeddings, the self-attention model, and the predictive output layer of the tokens to reduce the evaluated loss;
   (6) reiterating steps (1)-(5) until convergence of validation loss to a desired value;
(c) pretraining, by the one or more processors, the deep learning model to predict whether two breeder notes reference the same genotype, the pretraining constituting a next sentence prediction task comprising:
   (1) receiving by the deep learning model a first breeder token paired with one or more tokens for a breeder note, a separation token, and a second pair of breeder token and breeder note tokens that are sampled from notes referencing the same plant genotype;
   (2) performing with a frequency between 0 and 1, by the one or more processors, replacement of the second breeder, breeder note pair with an alternative pair referencing an different plant genotype from the first note;
   (3) converting all input tokens into token embeddings;
   (4) generating by the neural network a prediction of whether the first and second breeder note tokens are associated with the same plant genotype or different plant genotypes;
   (5) evaluating the loss function of the predicted association between notes with respect to its true value;
   (6) adjusting the weights of the token embeddings, the self-attention model, and the predictive output layer of the tokens to reduce the evaluated loss; and
   (7) reiterating steps (1)-(6) until convergence of validation loss to a desired value; and
(d) inputting a plurality of pairs of breeder tokens and breeder note tokens into the pre-trained deep learning model to generate a vector that corresponds to a unified embedding representation of a plurality of breeder notes for a particular plant genotype.

Embodiment 65. The method of embodiment 8, wherein the mask language modeling task comprises:
(a) inputting into the deep learning model one or more pairs of breeder tokens and their respective breeder note tokens for a specific plant genotype; and
(b) selecting a subset of the breeder note tokens to be evaluated by the loss function, wherein the subset of the breeder note tokens comprises a combination of breeder note tokens, masked breeder note tokens and/or masked breeder tokens, and randomly replaced breeder tokens and/or breeder note tokens.

Embodiment 66. A computer-implemented method for generating a unified representation for a plant genotype for one or more breeder's notes, the method comprising:
(a) receiving by a tokenizer implementing a tokenization scheme for a constructed vocabulary one or more breeder's notes, wherein the one or more breeder's notes comprise one or more word parts;
(b) assigning each word part of the one or more breeder's notes a token;
(c) assigning each breeder its own unique token;
(d) receiving by a deep learning model implementing self-attention in a computing device one or more pairings of breeder and breeder's notes, wherein the breeders are tokenized, and the breeder's notes comprise one or more word parts that have been encoded into tokens using a constructed vocabulary;
(e) converting by an embedding layer each token in the input to a unique token embedding corresponding to that token;
(f) pretraining the deep learning model to learn structural language patterns in the breeder notes, relationships of notes to breeders, and how notes co-occur with one another among plant genotypes, the pretraining constituting a masked language modeling task comprising:
   (1) performing selection of one or more tokens to be evaluated by a loss function following an output layer;
   (2) generating replacement of one or more of the selected input token embeddings from (1) with either an alternative token embedding selected from a tokenizer vocabulary or a token embedding representing the masked state;
   (3) generating by the deep learning model a prediction of the true token for each input token;
   (4) evaluating the loss function of the predicted tokens with respect to their true values for those tokens selected in (1);
   (5) adjusting the weights of the token embeddings, the deep learning self-attention model, and a predictive output layer of the tokens to reduce the evaluated loss;
   (6) reiterating steps (1)-(5) until convergence of validation the loss to a desired value; and
(g) inputting a plurality of pairs of breeder tokens and breeder note tokens into the pre-trained deep learning model to generate a vector that corresponds to a unified embedding representation of a plurality of breeder notes for a particular plant genotype.

Embodiment 67. The method of embodiment 66, the method further comprising:
(a) pretraining the deep learning model to learn associations between breeders notes and the breeder who wrote the notes, the pretraining comprising a next sentence prediction task comprising:
   (1) receiving by the deep learning model a breeder token paired with the one or more tokens for a breeder note from that breeder, converting all tokens into token embeddings;
   (2) performing with a frequency between 0 and 1, by the one or more processors, replacement of the true breeder token embedding with the breeder token embedding for an alternative breeder
   (3) generating by the deep learning model a prediction of whether the breeder token and breeder note tokens correspond to the true values;
   (4) evaluating a loss function of the predicted associations between the breeder tokens and breeder note token with respect to its true value;
   (5) adjusting the weights of the token embeddings, the deep learning self-attention model, and the predictive output layer of the tokens to reduce the evaluated loss;
   (6) reiterating steps (1)-(5) until convergence of validation the loss to a desired value;
(b) pretraining the deep learning model to predict whether two breeder notes reference the same genotype, the pretraining comprising a next sentence prediction task comprising:
   (1) receiving by the deep learning model a first breeder token paired with one or more tokens for a breeder note, a separation token, and a second pair of breeder token and breeder note tokens that are sampled from notes referencing the same plant genotype;
   (2) performing with a frequency between 0 and 1, by the one or more processors, replacement of the second breeder, breeder note pair with an alternative pair referencing an different plant genotype from the first note;
   (3) converting all input tokens into token embeddings;
   (4) generating a prediction of whether the first and second breeder note tokens are associated with the same plant genotype or different plant genotypes;
   (5) evaluating a loss function of the predicted association between notes with respect to its true value;
   (6) adjusting the weights of the token embeddings, the deep learning self-attention model, and the predictive output layer of the tokens to reduce the evaluated loss; and
   (7) reiterating steps (1)-(6) until convergence of validation the loss to a desired value.

Embodiment 68. The method of embodiment 67, where the pretraining step of (f) in embodiment 66 and the pretraining steps of embodiment 67 are performed simultaneously or sequentially or in combinations thereof.

Embodiment 69. The method of embodiment 66, wherein the mask language modeling task comprises:
(1) inputting into the deep learning model one or more pairs of breeder tokens and their respective breeder note tokens for a specific plant genotype; and
(2) selecting a subset of the breeder note tokens to be evaluated by a loss function, wherein the subset of the breeder note tokens comprises a combination of breeder note tokens, masked breeder note tokens and/or masked breeder tokens, and randomly replaced breeder tokens and/or breeder note tokens.

Embodiment 70. The method of embodiment 66, wherein the generated vector from step (g) is used to facilitate advancement decisions or to predict an advancement score.

Embodiment 71. The method of embodiment 67, wherein in step (a), the one or more breeder tokens is paired with a breeder note token for the correct/actual breeder.

Embodiment 72. The method of embodiment 67, wherein in step (a), the one or more breeder tokens is paired with a breeder note token for the breeder who did not write the note, where the breeder is incorrect.

Embodiment 73. The method of embodiment 67, wherein in step (b), the breeder token is the same (for the same breeder).

Embodiment 74. The method of embodiment 67, wherein in step (b), the breeder token is different (for a different breeder).

Embodiment 75. The method of embodiment 67, wherein in step (b), the first and second breeder note tokens are from the same breeder token but are associated with the different plant genotypes.

Embodiment 76. The method of embodiment 67, wherein in step (b), the first and second breeder note tokens are from the different breeder tokens and are associated with the different plant genotypes.

Embodiment 77. The method of embodiment 67, wherein in step (b), the first and second breeder note tokens are from the different breeder tokens but are associated with the same plant genotypes.

Embodiment 78. The method of embodiment 67, wherein in step (b), the first and second breeder note tokens are associated with the same plant genotype.

Embodiment 79. The method of embodiment 67, wherein in step (b), the first and second breeder note tokens are from the same breeder but for a different plant genotype.

Embodiment 80. The method of embodiment 66, wherein the true token is a true breeder note token or a true breeder token.

Embodiment 81. The method of embodiment 66, wherein the true grouping values indicate whether the breeder notes are for the same plant genotype.

Embodiment 82. The method of embodiment 66, wherein the mask language modeling task comprises:
masking a certain percentage of the subset of the breeder note tokens and/or breeder tokens to create breeder note tokens and/or breeder tokens.

Embodiment 83. The method of any of the preceeding embodiments, wherein the generated vector from embodiment 66 (step g) is used as input in a machine learning model or model to train the machine learning model.

Embodiment 84. The method of any of the preceeding embodiments, wherein the generated vector from embodiment 66 (step g) is used as input in a machine learning model or model train the machine learning model to predict an advancement score for a candidate plant genotype.

Embodiment 85. The method of any of the preceeding embodiments, wherein the generated vector from step (g) of claim 66 is used as input in a model to train the machine learning model or model to generate an advancement score for a candidate plant genotype, wherein the particular plant genotype is a parent or derivative of the candidate plant genotype.

Embodiment 86. The method of any of the preceeding embodiments, wherein the generated vector from step (g) of claim 66 is used as input in a model to facilitate advancement decisions or to predict an advancement score.

Embodiment 87. The method of embodiment 66, wherein the weights of the token embeddings, and/or the self-attention model of the deep learning model, and/or the predictive output layer of the tokens are adjusted to weight breeders notes for a particular geographic region, breeding program, or targeted set of environments.

Embodiment 88. The method of any of the preceeding embodiments, wherein the generated vector from step (g) of claim 66 is used as input in a model to facilitate advancement decisions, wherein the particular plant genotype is from or for an inbred, hybrid, doubled haploid, plant from a doubled haploid, or a cross or derivative thereof.

Embodiment 89. A system comprising:
(a) one or more servers, each of the one or more server storing plant data; and
(b) a computing device communicatively coupled to the one or more servers, the computing device including:
   (1) a memory; and
   (2) one or more processors configured to perform operations comprising:
      (a) obtain one or more breeder's notes, wherein the breeder's notes comprise one or more word parts that have been encoded into tokens using a constructed vocabulary;
      (b) assign each breeder its own unique token;
      (c) pretrain a deep learning model to learn structural language patterns in the breeder notes, relationships of notes to breeders, and how notes co-occur with one another among plant genotypes;
         (1) mask and/or randomly replace a plurality of selected breeder tokens and breeder note tokens;
         (2) predict a true token foreach input breeder token and breeder note token;
         (3) evaluate a loss function of the predicted tokens with respect to their true values;
         (4) adjust the weights of the token embeddings, the self-attention model, and the predictive output layer of the tokens to reduce evaluated loss;
      (d) receive a plurality of pairs of breeder tokens and breeder note tokens into the pre-trained deep learning model; and
      (e) generate a vector that corresponds to a unified embedding representation of a plurality of breeder notes for a particular plant genotype.

Embodiment 90. The system of embodiment 89, wherein the one or more processors are configured to perform the operations comprising:
(f) pretrain the deep learning model to predict for a first given next-sentence prediction task whether a plurality of breeder notes is associated with the breeder who wrote the note;
   (1) receive one or more breeder tokens paired with a breeder note token for a specific plant genotype in a neural network of the deep learning model;
   (2) replace, with frequency between 0 and 1, the true breeder token with an alternative breeder note token;
   (3) generate a prediction whether the breeder token and breeder note tokens are correctly paired;
   (4) evaluate a loss function of the predicted associations between the breeder tokens and breeder note tokens with respect to its true values;
   (5) adjust the weights of the token embeddings, the self-attention model, and the predictive output layer of the tokens to reduce the evaluated loss;
   (6) reiterate steps (1)-(5) until convergence of the validation loss;
(g) pretrain the deep learning model to predict whether two breeder notes are associated with the same plant genotype or different plant genotypes;
   (1) receive into the deep learning model a breeder token paired with one or more first breeder note tokens for a first plant genotype, a separation token, and a breeder token paired with one or more second breeder note tokens for a plant genotype that is the same as the first plant genotype or different than the first plant genotype;
   (2) generate a prediction of whether the first and second breeder note tokens are associated with the same plant genotype or different plant genotypes;
   (3) evaluate a loss function of the predicted associations between the plant breeder token and breeder note token with respect to its true grouping values;
   (4) adjust the weights of the token embeddings, the self-attention model, and the predictive output layer of the tokens to reduce the evaluated loss; and
   (5) reiterate steps (1)-(4) until convergence of the validation the loss.

Embodiment 91. The system of embodiment 90, wherein the one or more processors are configured to perform the operations comprising:
pretrain as set forth in step (c) in embodiment 89 and steps (f) and (g) in embodiment 90 simultaneously or sequentially or in combinations thereof.

Embodiment 92. A computer-implemented method for generating a unified representation for a plant genotype for one or more breeder's notes, the method comprising:
(a) receiving by a deep learning model implementing self-attention in a computing device one or more breeder's notes, wherein the breeder's notes comprise one or more word parts that have been encoded into tokens using a constructed vocabulary;
(b) assigning each breeder its own unique token;
(c) pretraining, by the one or more processors, the deep learning model to learn structural language patterns in the breeder notes, relationships of notes to breeders, and how notes co-occur with one another among plant genotypes;
(d) pretraining, by the one or more processors, the deep learning model to predict, for a first given next-sentence prediction task whether a plurality of breeder notes is associated with the breeder who wrote the note;
(e) pretraining, by the one or more processors, the deep learning model to predict, for a second given next-sentence prediction task whether the breeder note is associated with the same genotype or a different genotype; and
(f) inputting a plurality of pairs of breeder tokens and breeder note tokens into the pre-trained deep learning model to generate a vector that corresponds to a unified/single representation of a plurality of breeder notes for a particular plant genotype.

Embodiment 93. A system comprising:
(a) one or more servers, each of the one or more server storing plant data; and
(b) a computing device communicatively coupled to the one or more servers, the computing device including:
   (1) a memory; and
   (2) one or more processors configured to perform operations comprising:
      (a) obtain one or more breeder's notes, wherein the breeder's notes comprise one or more word parts that have been encoded into tokens using a constructed vocabulary;
      (b) assign each breeder its own unique token;
      (c) pretrain the deep learning model to learn structural language patterns in the breeder notes, relationships of notes to breeders, and how notes co-occur with one another among plant genotypes;
         (1) mask and/or randomly replace a plurality of selected breeder tokens and breeder note tokens;
         (2) predict a true (observed) token for each input breeder token and breeder note token;
         (3) evaluate the loss function of the predicted tokens with respect to their true values;
         (4) adjust the weights of the token embeddings, the self-attention model, and the predictive output layer of the tokens to reduce evaluated loss;
      (d) pretrain the deep learning model to predict for a first given next-sentence prediction task whether a plurality of breeder notes is associated with the breeder who wrote the note;
         (1) receive one or more breeder tokens paired with a breeder note token for a specific plant genotype in the neural network of the deep learning model;
         (2) replace, with frequency between 0 and 1, the true (observed) breeder token with an alternative breeder note token;
         (3) generate a prediction whether the breeder token and breeder note tokens are correctly paired
         (4) evaluate the loss function of the predicted associations between the breeder tokens and breeder note tokens with respect to its true values;
         (5) adjust the weights of the token embeddings, the self-attention model, and the predictive output layer of the tokens to reduce the evaluated loss;
         (6) reiterate steps (1)-(5) until convergence of the validation loss;
      (e) pretrain the deep learning model to predict whether two breeder notes are associated with the same genotype or different genotypes;
         (1) receive into the deep learning model a breeder token paired with one or more first breeder note tokens for a first plant genotype, a separation token, and a breeder token paired with one or more second breeder note tokens for a genotype that is the same as the first plant genotype or different than the first plant genotype;
         (2) generate a prediction of whether the first and second breeder note tokens are associated with the same plant genotype or different plant genotypes;
         (3) evaluate the loss function of the predicted associations between the plant breeder token and breeder note token with respect to its true grouping values;
         (4) adjust the weights of the token embeddings, the self-attention model, and the predictive output layer of the tokens to reduce the evaluated loss; (5) reiterate steps (1)-(4) until convergence of the validation loss;
      (f) receive a plurality of pairs of breeder tokens and breeder note tokens into the pre-trained deep learning model; and
      (g) generate a vector that corresponds to a unified embedding representation of a plurality of breeder notes for a particular plant genotype.

### Embodiment 94

A system comprising:
(a) one or more servers, each of the one or more server storing plant data; and
(b) a computing device communicatively coupled to the one or more servers, the computing device including:
   (1) a memory; and
   (2) one or more processors configured to perform operations comprising:
      (a) obtain one or more breeder's notes, wherein the breeder's notes comprise one or more word parts that have been encoded into tokens using a constructed vocabulary;
      (b) assign each breeder its own unique token;
      (c) pretrain the deep learning model to learn structural language patterns in the breeder notes, relationships of notes to breeders, and how notes co-occur with one another among plant genotypes;
      (d) pretrain the deep learning model to predict, for a first given next-sentence prediction task whether a plurality of breeder notes is associated with the breeder who wrote the note;
      (e) pretrain the deep learning model to predict whether two breeder notes are associated with the same or different genotypes; and
      (f) receive as input a plurality of pairs of breeder tokens and breeder note tokens into the pre-trained deep learning model to generate a vector that corresponds to a unified embedding representation of a plurality of breeder notes for a particular plant genotype.

Embodiment 95. The method of any of the preceeding embodiments, wherein the plant genotype or candidate plant genotype is for a monocot or dicot plant.

Embodiment 96. The method of any of the preceeding embodiments, wherein the plant genotype or candidate plant genotype is for a soybean, maize, sorghum, cotton, canola, sunflower, rice, wheat, sugarcane, alfalfa tobacco, barley, cassava, peanuts, millet, oil palm, potatoes, rye, or sugar beet plant.

Embodiment 97. The system of any of the preceeding embodiments, wherein the plant genotype or candidate plant genotype is for a monocot or dicot plant.

Embodiment 98. The system of any of the preceeding embodiments, wherein the plant genotype or candidate plant genotype is for a soybean, maize, sorghum, cotton, canola, sunflower, rice, wheat, sugarcane, alfalfa tobacco, barley, cassava, peanuts, millet, oil palm, potatoes, rye, or sugar beet plant.

### Examples

The present disclosure is further illustrated in the following Examples. It should be understood that these Examples, while indicating embodiments of the invention, are given by way of illustration only. Thus, various modifications to the types of machine learning models, learned breeding strategies, and their use in advancement decisions, and breeding are disclosed.

### EXAMPLE 1: A Machine Learning Model for Learning a Breeding Strategy of a Retrospective Index

In one embodiment, one of the machine learning models uses a retrospective selection index as described by Bernardo (1991). The index weights are found by comparing the normalized trait values of the selected candidates to the normalized trait values of the complete set of candidates to determine the selection differential [s] the breeder created with their selections. The selection differential is then multiplied by the inverse of the variance-covariance matrix [C⁻¹] of the normalized trait values to account for the phenotypic covariance in the set of candidate lines. The result of this calculation is a selection index [b]. This procedure is expressed as:

$$b = {C}^{- 1} s$$

Multiplying the normalized MLE predicted trait values of a candidate [xₚ] by this index [b] quantifies the overall net likelihood of advancement of the line as a dot product of its predicted traits and the retrospective index weight for those traits:

$${merit}_{A} = b {x}_{p}$$

### EXAMPLE 2: A Machine Learning Model for Learning a Breeding Strategy of a Multivariate Gaussian Distribution

In one embodiment, one of the machine learning models fits a multivariate Gaussian distribution to the normalized trait predictions of the selected lines in the decision dataset. Traditionally, multivariate Gaussian probability density is expressed as:

$${( 2 π )}^{- \frac{k}{2}} \cdot {det ( {C}_{s} )}^{- \frac{1}{2}} \cdot {e}^{- \frac{1}{2} {( x - μ )}^{T} {C}_{s}^{- 1} ( x - μ )}$$

When working with a fixed set of MLE estimates and standard normal transformed trait predictions, this can be simplified to:

$$c \cdot {e}^{\frac{- {x}^{T} {C}_{s}^{- 1} x}{2}}$$

Where [c] is a normalizing constant, [x] is a vector of predicted normalized trait values, and [Cₛ⁻¹] is the inverted covariance matrix of trait values for the selected lines. The result of this expression is the relative likelihood that a line would be selected given its normalized trait values [x].

$${merit}_{B} = c \cdot {e}^{\frac{- {x}_{p}^{T} {C}_{s}^{- 1} {x}_{p}}{2}}$$

### EXAMPLE 3: A Machine Learning Model for Learning a Breeding Strategy of a Univariate Normal Distribution

In one embodiment, one of the machine learning models fits a univariate normal distribution to the normalized harvest grain moisture predictions of the selected lines in the decision dataset. This probability density is expressed as:

$$\frac{1}{σ \sqrt{2 π}} {e}^{- \frac{1}{2} {( \frac{x - μ}{σ} )}^{2}}$$

Because the grain moisture predictions are normalized to mean zero and unit variance, this can be simplified to:

$${ce}^{- \frac{1}{2} {x}^{2}}$$

Where [c] is a normalizing constant and [x] is the predicted normalized harvest grain moisture. The result of this expression is the relative likelihood that a line would be selected given its normalized harvest grain moisture.

$${Merit}_{C} = {ce}^{- \frac{1}{2} {x}^{2}}$$

### EXAMPLE 4: A Ensemble of Machine Learning Models

The final ensembled prediction combines the results of each component model to produce an advancement score for each candidate line. Output from Model A disclosed in Example 1 and Model B disclosed in Example 2 are rescaled to mean zero and unit variance, while the output from Model C disclosed in Example 3 is left unscaled to represent a relative likelihood.

$${Merit}_{N ( A )} = \frac{{Merit}_{A} - {μ}_{A}}{{σ}_{A}}$$

$${Merit}_{N ( B )} = \frac{{Merit}_{A} - {μ}_{A}}{{σ}_{A}}$$

The final score is a described by the expression:

$${Score}_{L B S} = ( {Merit}_{N ( A )} \cdot 0.8 + {Merit}_{N ( B )} \cdot 0.2 ) \cdot \sqrt{{Merit}_{C}}$$

Anecdotal evidence suggests that Models A and B complement each other, with Model A performing better in many cases, and Model B performing better in a few cases where Model A performs poorly. The rescaled output of Models A and B are averaged using an 80/20 ratio to address this anecdote.

The output of model C is based on the harvest grain moisture, which is highly correlated with the maturity of experimental lines. The result of this multiplication is that the intermediate score from Models A and B is restricted to lines where the harvest grain moisture and thus the maturity of the lines matches those advanced in the past. Breeders are often limited by the maturity of the lines they can bring to market. Lines with excellent breeding likelihood of advancement may be discarded because they mature too early or too late for the breeder's target environment. The square root operation flattens the probability curve, intentionally biasing the ensemble towards overscoring outliers rather than underscoring borderline candidate lines.

### Learning a Breeding Strategy

All lines within a decision dataset were predicted for all traits that their breeding team provided that could be of interest. The predicted trait values were scaled to mean zero and unit variance, and the scaling coefficients were recorded. The predicted decision dataset was used to fit all three component models Model A, Model B, and Model c disclosed in Examples 1, 2, and 3 respectively.

1. The retrospective index in Example 1 was found for the predicted decision dataset using the normalized predicted trait values for the lines, and the retrospective index was recorded. The lines in the decision dataset were scored using the retrospective index, and the mean and variance of the scores were recorded.

2. The multivariate gaussian parameters from Example 2 were fit to the normalized predicted traits for only the selected lines within the decision dataset. The covariance matrix and rescaling constant of the multivariate gaussian probability density function was recorded. The lines of the decision dataset were scored using the multivariate gaussian model, and the mean and variance of the scores were recorded.

3. The maturity distribution model parameters from Example 3 were fit to the normalized harvest grain moisture predictions as a proxy for relative maturity.

These values alongside the MLEs used to generate the predictions on the decision dataset constitute the learned breeding strategy.

### Using a Learned Breeding Strategy

A new set of candidate lines, candidate plant genotypes, was identified that had not previously been considered by the breeder. The lines were predicted for all traits using MLEs. The lines were predicted for their genetic value, and the predictions were scaled using the scaling coefficients used to normalize the decision dataset. Then the lines were scored using the three trained component machine learning models and the final ensembled prediction, advancement score, was made for each line.

1. The retrospective index was computed on the rescaled candidate line predictions, then the resulting index values were rescaled using the mean and variance from the decision dataset's retrospective index scores.

2. The multivariate gaussian index was computed on the rescaled candidate line predictions, then the resulting index values were rescaled using the mean and variance from the decision dataset's gaussian index scores.

3. The maturity distribution model was computed on the rescaled candidate line harvest grain moisture predictions.

4. The ensemble model prediction was made from the three individual machine learning model advancement scores for every candidate line.

### EXAMPLE 5: Evaluation

The model was evaluated both quantitatively and qualitatively to demonstrate effectiveness.

### Qualitative Evaluation

The primary purpose of the qualitative evaluation was to determine if learned breeding strategies were consistent across years and among breeders within a similar geography and target market. Learned breeding strategies were prepared from each of a variety of decision datasets spanning multiple breeders, multiple selection decisions, and multiple years totaling 13 decisions. All 13 breeders target the North America hybrid corn market with maturity between 113 and 118 CRM. An independent set of candidate lines was scored using all 13 learned breeding strategies, and the average score for the 13 strategies was computed on each line. The lines with the smallest and largest average scores were evaluated to observe the similarity of breeding strategies (FIGS. 12A and 12B).

### Quantitative Evaluation

The purpose of the quantitative evaluation was to determine the if learned breeding strategies were consistent across multiple years of decision datasets for the same breeder. A pair of decision datasets were collected from the same breeder for the same stage of the Corteva inbred maize advancement pipeline representing the same decision made on different candidates in different years. A learned breeding strategy was learned for each year's decision, then the learned strategies were utilized with the lines from the alternate year. We observed the proportion of advanced lines that had learned breeding strategy scores that were greater than zero, indicating they scored above average on a different year's learned strategy.

TABLE 1
Overlap between actual selected candidates and candidates
scoring above average for learned breeding strategies. At
least 95% of advanced lines scored above average, indicating
that the learned breeding strategies assigned a positive
score to most lines the breeder chose to advance.

| | Candidate | | | |
|---|---|---|---|---|
| Strategy | Lines | Actual | Selections | Selections |
| Year | Year | Selections | Scoring > 0 | Scoring < 0 |
| 2019 | 2018 | 381 | 361 (95%) | 20(5%) |
| 2018 | 2019 | 469 | 454 (97%) | 15 (3%) |

### EXAMPLE 6: A Machine Learning Model for Learning a Breeding Strategy with Coancestry Penalty

A penalty score may be calculated based on the pairwise coancestry between a set of candidate lines scored by the learned breeding strategy, wherein the learned breeding strategy could be any embodiment producing an advancement score. For example, in FIG. 5, the penalty score model could be defined such that it reduces the overall advancement score of solutions with coancestry greater than a threshold value. Suppose we have a function “CoA” to compute the coancestry between pairs of lines selected by a candidate learned breeding strategy model. The “CoA” function returns a value between 0 and 1, where 0 is unrelated and 1 is genetically identical. A penalty multiplier could be defined as follows.

$$AverageCoancestry = {\sum }_{i = 1}^{n} {\sum }_{j = 1}^{n} \frac{CoA ( i , j )}{{n}^{2}}$$

$$ExcessCoA = AverageCoA - TargetCoA$$

$$Penalty = \{ \begin{matrix}ExcessCoA if ExcessCoA > 0 \\ 0 otherwise\end{matrix}$$

$$ScoreMultiplier = 1 - λ * Penalty$$

This multiplier may be applied to the advancement score output of any machine learning model, deep learning model, or ensemble thereof. The result is that solutions with coancestry below the TargetCoA are classified as sufficiently diverse and do not receive a penalty. Solutions with coancestry above the TargetCoA are considered too closely related and receive a penalty proportional to the amount of excess coancestry they possess above the target value. The magnitude of the penalty is adjustable by an additional parameter, 2. Alternative diversity metrics such as effective population size or parent use counts may be substituted in place of the coancestry metric.

### Quantitative Evaluation

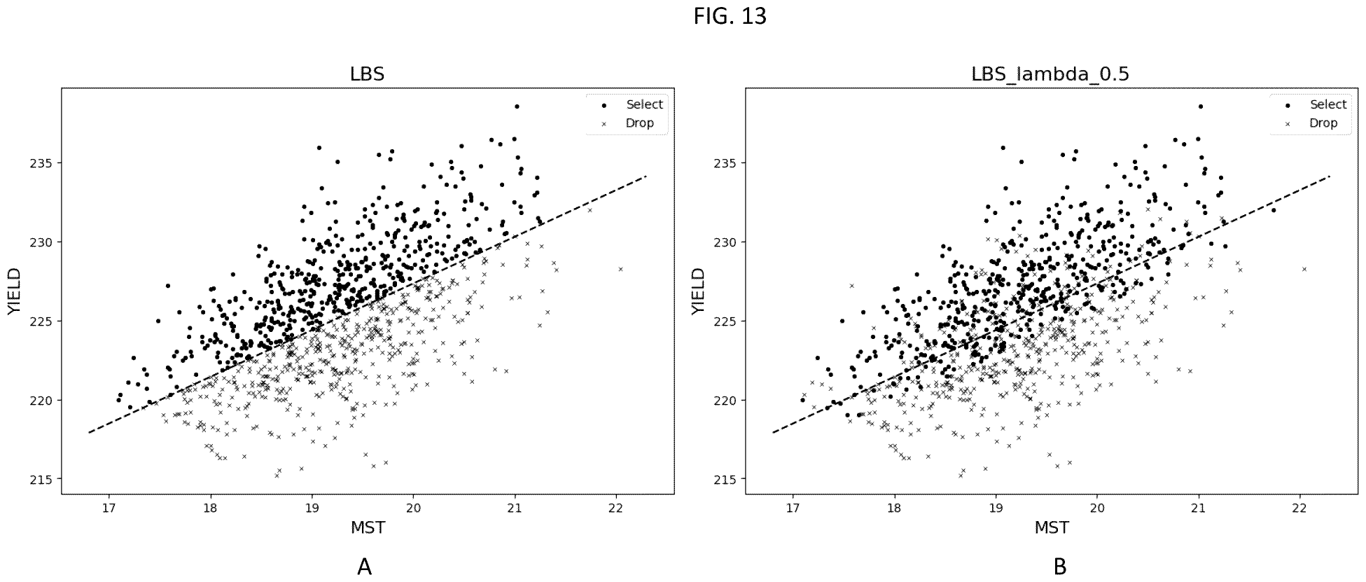

A simulation study was undertaken to assess the impact of imposing coancestry penalties of varying strengths on a learned breeding strategy based on grain yield and moisture values. With a selection intensity of 50% and a coancestry penalty of zero, only genotypes above the LBS weight combination of traits were selected (FIG. 13). However, with the imposition of a positive coancestry penalty, a minority of genotypes in the unpenalized selected cohort were replaced with entries having a smaller trait-driven advancement score but less average coancestry with the rest of the selected entries.

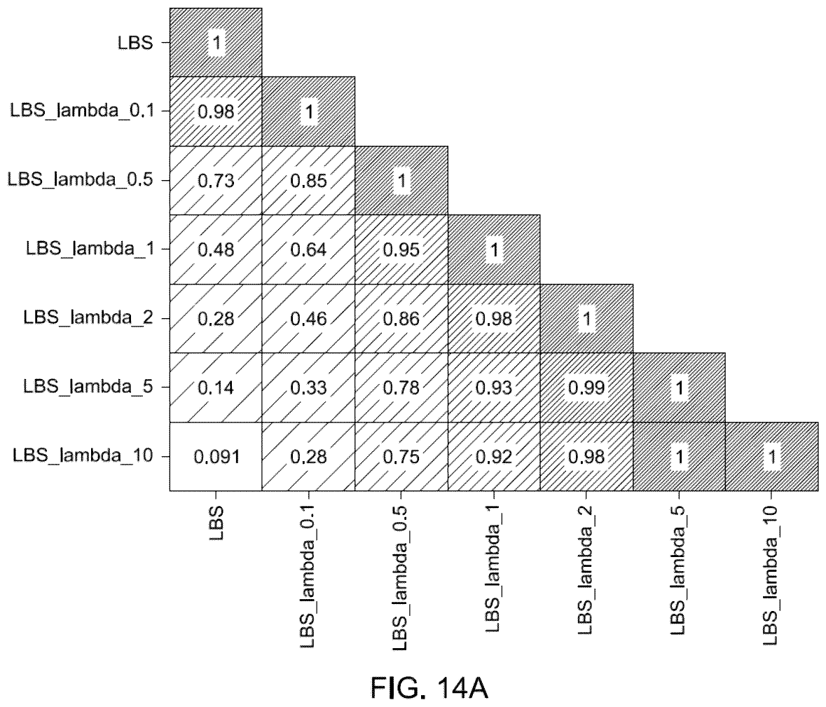

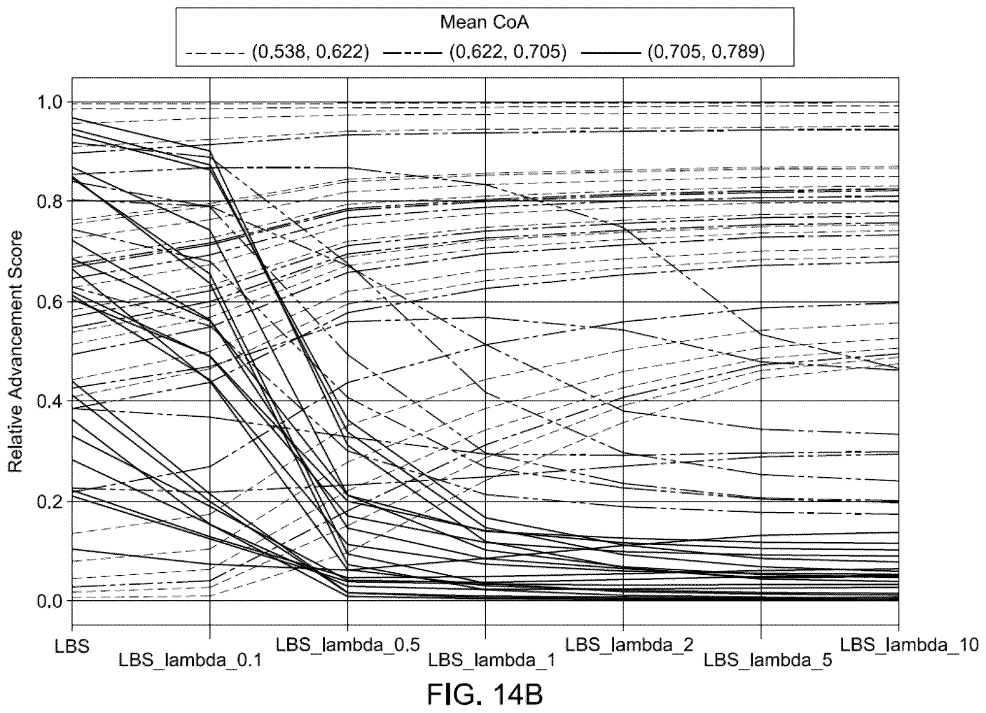

As the coancestry penalty is raised from the zero, the overall LBS score is less driven by trait scores and more driven by minimization of relatedness among selected genotypes (FIG. 14). Entries with the highest average of coancestry with the original cohort shift ranks more quickly to an overall score, while entries with little coancestry to the selected cohort increase their relative scores (FIG. 14).

### EXAMPLE 7: Deep Learning Prediction of Breeder Preferences from Historical Data

In addition to or instead of training a separate advan model for each breeder, a meta-prediction approach may be employed that leverages both breeder-specific and cross-program breeder preferences within the context of the available germplasm for that year. This type of model requires at least three types of input: 1) a representation of the candidate genetics that permits evaluation of the relevant phenotypes, 2) a representation of the breeding target environment for which to make the predictions, and 3) a summarization of the full germplasm set under consideration within the program. The third type of input accounts for the non-stationary nature of the prediction problem, due to the influence of genetic gain with each breeding cycle. Deep neural networks provide a flexible means of combining such disparate and high-dimensional types of information for predictions.

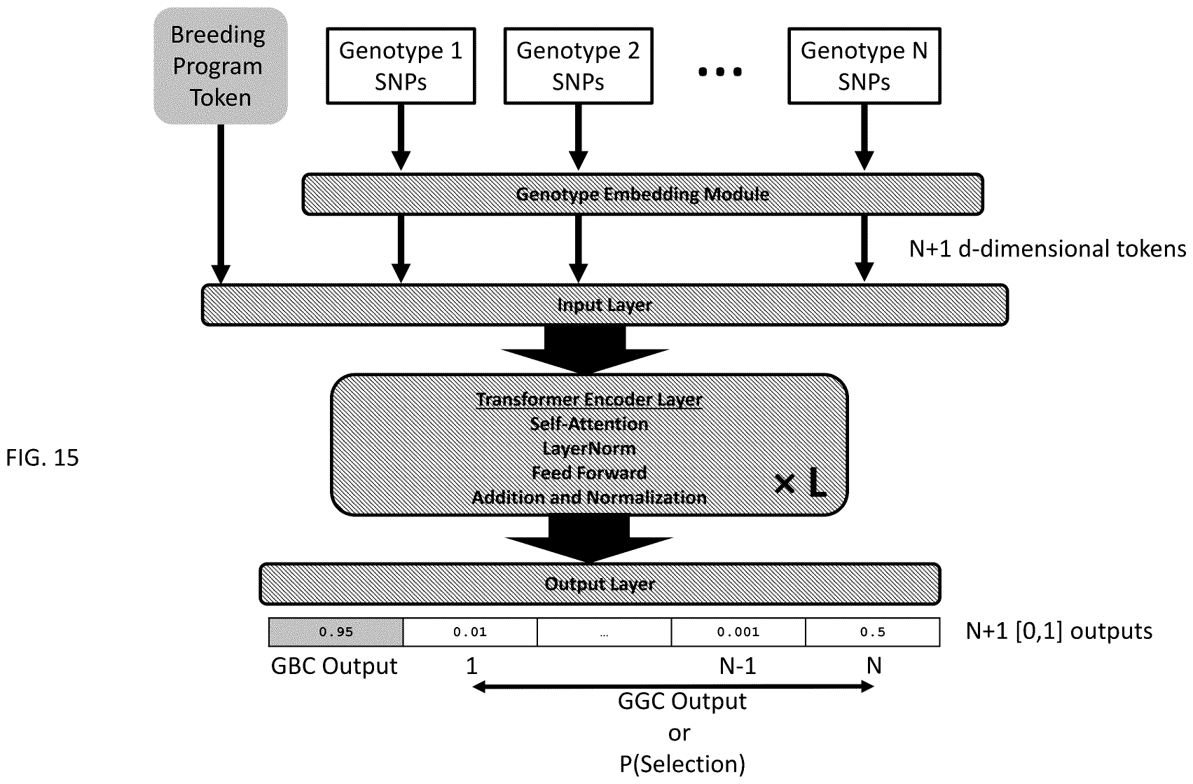

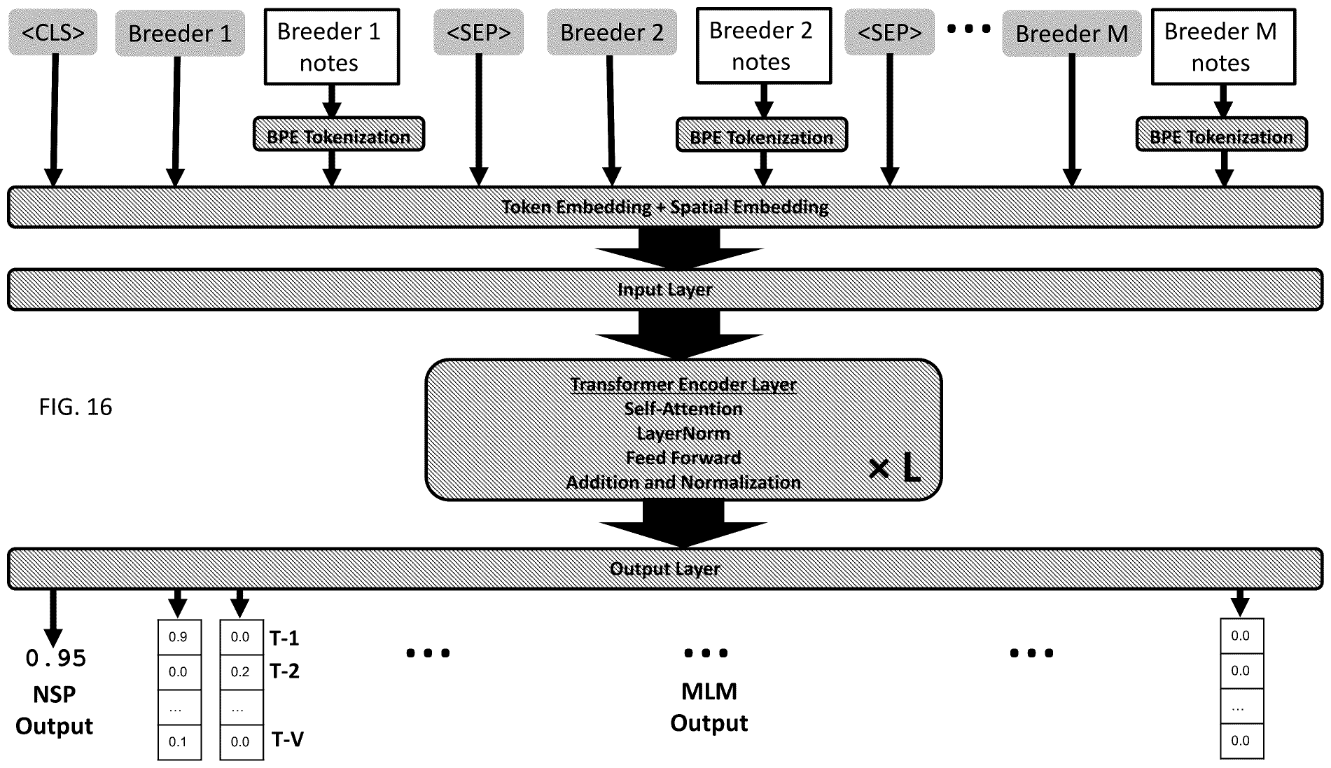

The inputs to this neural network consist of d-dimensional vectors of real numbers, hereafter referenced as tokens (FIG. 15). For this problem, we make use of two token types: 1) genotype tokens conditioned on the genetics of the hybrid or inbred line under consideration, and 2) breeding target environment tokens acting as embedding vectors for each program under consideration. Inputs to the neural network consist of multiple genotype tokens and a single breeding target environment token. The use of multiple genotype tokens permits contextualization of each genotype within the full population of genotypes input into the network, while the breeding target environment token adds the context of the breeding target environment in which selection is to be conducted. While the breeding target environment tokens are learned without conditioning on any other variables, genotype tokens are learned embeddings conditioned on genotypic data. For instance, latent representations derived from variational encoders conditioned on SNP data is processed by one or more dense feed-forward layers before output as the final d-dimensional token.

Transformer-based neural networks benefit highly from an initial self-supervised pre-training_stage, wherein the contextual patterns may be learned even the absence of additional labeled data. For this problem case, the pre-training tasks are oriented toward two desired outcomes. First, the network should encode how different genotypes co-occur with one another over space and time. Second, the network should encode the correspondence between genotypes and breeding target environments. To achieve the first goal, we train with the genotype-genotype context (GGC) task. For this task, N-M genotypes are sampled from a single historical location for a single experiment within that location. Another M genotypes are sampled either from the same location and experiment or at random from other locations and experiments. For these M genotypes, the target output is a binary classification of whether each is sampled from the same location and experiment as the first N-M genotypes. For the second task, genotype-breeder context (GBC), a single binary target is provided indicating whether the location and experiment of the N-M genotypes corresponds to the provided breeding target environment token, which will be sampled at random among breeding target environments with probability p. Both pre-training tasks are trained simultaneously using separate head layers with a cross-entropy loss function.

Following pre-training across thousands of historical locations, the head and lower layers of the encoder network are fine-tuned for the task of predicting the probability of selection within each breeding target environment. Training consists of presenting the encoder with a sample of candidate genotypes along with the token for their corresponding breeding target environments. Target outputs for each candidate genotype are provided as 0/1 values, based on whether any given genotype was historically selected. Training proceeds with binary outputs from the head layer and a cross-entropy loss function.

Following training, prediction of likelihood of advancement proceeds by feeding the breeding target environment token embedding and the set of candidate genotype token embeddings to the neural network. Sigmoid-transformed outputs from the head layer represent the learned probability that each genotype will be selected by the specified breeding target environment. Because the computational complexity of prediction scales quadratically with the number of candidate genotypes under our prediction architecture, one may use a sampling approach, wherein each genotype is evaluated within the context of a random subset from the candidate set. Averaging of such sampled predictions thereby provides an ensembling mechanism for reducing prediction error.

### EXAMPLE 8: Incorporating breeder notes as input into Learned Breeding Strategies

Although the traditional agricultural traits (e.g. grain yield, moisture, plant height) are all primary considerations during the development of breeding strategies, breeders also take extensive field notes that may be used to inform crossing and advancement decisions. Unlike trait values, field notes do not readily lend themselves to numerical approaches. They lack the standardized structure of field trait data, and the form of these notes is highly idiosyncratic to each breeder. In order to allow feedback from breeder field notes to inform learned breeding strategies, one may use natural language processing (NLP) approaches that convert notes into standard numerical representations. The NLP models process the language and place the notes within the context of of the breeder who wrote them. Following the embedding of the breeder notes, they may act as input to multiple learned breeding strategies methodologies.

As described in Example 7, modern transformer-based architectures are suitable for placing breeding data within the context of a given breeding target environment. A multi-layer transformer-based encoder model may be developed for transforming breeder notes into a contextualized embedding by pre-training it on two self-supervised tasks—the first a variant of the masked language modelling (MLM) task and the second a variant of the next sentence prediction (NSP) task.

Prior to pre-training, a tokenization protocol for breeder notes must be specified. The development of a byte-pair encoding vocabulary of breeder notes proceeds by first allotting a token for each character among all breeder notes, then recursively combining the most common pairs of tokens in the breeder note corpus into single tokens until a target vocabulary size is reached. Each unique breeder is also assigned their own token, as these breeder tokens will be paired with the tokenizations of the notes during input into the encoder model. Special tokens indicating <START>, <STOP>, <September>, <CLS>, and <MASK> are also included.

Pre-training consists of two self-supervised tasks-MLM and NSP. The MLM task serves as the primary means for allowing the model to learn structural language patterns in the notes, relationships of notes to breeders, and how notes co-occur with one another among genotypes. The NSP task may be used to augment learning of how specific breeders and notes are related, along with how they co-occur among different genetics.

In the MLM task, input consists of one or more coupled breeders and breeder notes for a single genotype. If multiple breeder notes are provided, the (breeder, note) pairs are separated by <September> tokens in the input. A minority of tokens (e.g. 15%) are chosen at random to inform the calculation of loss for each example. Within the input, 80% of these chosen tokens are replaced with a <MASK> token, 10% are replaced with a random alternative token from the vocabulary, and 10% are retained as-is. The input tokens are embedded as d-dimensional input vectors, to which a set of spatial encoding vectors is added in order to preserve relative ordering information throughout the self-attention layers. A softmax output head layer on top of the encoder provides the current prediction of the true output token, and the loss may then be computed using a cross-entropy function. Two related variants of the NSP task may also be used during pretraining. In both, the loss is based on a binary output, with a single-output head layer placed beneath the output token corresponding to the <CLS> input. In the first NSP task, a breeder token is either coupled with a note from that breeder or with a random note from another breeder, with loss based on the ability of the model to predict correct versus incorrect pairings. In the second NSP task, an initial (breeder, note) pair is given, along with a second (breeder, note) pair from either the same genotype or from a random chosen genotype. Again, loss is calculated based on the ability to predict whether the notes corresponded to the same genotype.

Following training, embeddings of notes may be derived from either the encoder output corresponding to the <CLS> token or taken from an averaging of the encoder outputs of all input tokens. These embeddings may then be used as additional inputs to other learned breeding strategies prediction techniques. For example, the embeddings of notes may be arithmetically added to the input embeddings of genotypes within the structure of the deep learning model described in Example 7. They may also be provided as inputs analogous to phenotypic traits for the types of non deep-learning models described in Examples 1-6.

1. A computer-implemented method for use in plant breeding comprising:
(a) receiving input data comprising data from candidate plant genotypes being considered for advancement through a computing device;
(b) inputting candidate data comprising data from candidate plant genotypes being considered for advancement into the ensemble of the at least two trained machine learning models, wherein the at least two trained machine learning models have been trained to learn a likelihood of advancement of a plant; and
(c) generating by the ensemble an advancement score for each candidate plant genotype.

2. A computer-implemented method for use in plant breeding comprising:
(a) inputting into a pre-trained deep learning model in a computing device a plurality of candidate plant genotypes that a breeding target environment is considering along with the breeding target environment token that is considering the plurality of candidate plant genotypes to generate an advancement score for each plant genotype.

3. The method of claim 1, the method further comprising training the ensemble by
(a) receiving, through one or more computing devices, at least one training data set comprising data from a breeder's selections of plants for advancement;
(b) inputting the data from the at least one training data set into an ensemble of at least two machine learning models;
(c) training the ensemble of the at least two machine learning models to learn a likelihood of advancement of a plant genotype from the training data set;
(d) inputting candidate data comprising data from candidate plant genotypes being considered for advancement into the ensemble of the at least two trained machine learning models; and
(e) generating by the ensemble an advancement score for each candidate plant genotype.

4. The method of claim 2, the method further comprising:
(a) receiving by a deep learning model implementing self-attention in a computing device one or more plant genotype representations and one or more representations of breeding target environments associated with the training plant genotype representations;
(b) simultaneously learning by the deep learning model an association among genotypes and between plant genotypes and breeding target environments to produce a prediction whether plant genotypes are associated with one another and with a given breeding target environment;
(c) evaluating a loss function of the predicted associations among the plant genotypes and predicted associations among the plant genotypes and breeding target environments with respect to their true grouping values;
(d) adjusting the weights of the embeddings, the self-attention model, and/or the predictive output layer of the tokens or combinations thereof to reduce the evaluated loss; and
(e) reiterating steps (a)-(d).

5. The method of claim 2, the method further comprising fine tuning by
(a) receiving by the pre-trained deep learning model implementing self-attention in a computing device one or more plant genotype representations and one or more representations of breeding target environments associated with the plant genotype representations, wherein the plant genotype representations comprise token embeddings and plurality breeding target environment representations are tokens to produce a predicted advancement score;
(b) evaluating a loss function of the predicted advancement score for each plant genotypes with respect to their true advancement values;
(c) adjusting one or more of the weights of the token embeddings, the self-attention model, and the predictive output layer of the tokens to reduce the evaluated loss; and
(d) reiterating steps (a)-(c).

6. The method of claim 2, the method further comprising:
selecting one or more candidate plant genotypes based on its advancement score.

7. The method of claim 6, the method further comprising:
growing one or more of the selected candidates.

8. The method of claim 7, the method further comprising:
obtaining a tetrad microspore from the one or more selected candidates;
contacting the tetrad microspore with a chromosome doubling agent to produce a doubled haploid embryo; and
generating a doubled haploid plant from the doubled haploid embryo.

9. The method of claim 7, the method further comprising:
crossing one or more of the selected candidates with (1) a maternal inducer line to produce seeds with haploid embryos, (2) itself to create an improved inbred population having desirable (improved) characteristics, or (3) another candidate or breeding plant to create an improved offspring (hybrid) with desirable (improved) characteristics, improved hybrid vigor, or combinations thereof.

10. The method of claim 4, wherein the representations of plant genotypes and representations of the breeding target environments are tokens with vector embeddings.

11. The method of claim 1, the method further comprising: generating by the ensemble an advancement score for each candidate plant genotype for a particular environment or region and/or for a particular characteristic/trait.

12. The method of claim 2, the method further comprising: generating an average advancement score from two or more breeders for each candidate plant genotype.

13. The method of claim 2, wherein the method comprises selecting a subset of candidate plant genotypes from a plurality of candidate plant genotypes based on the advancement scores of the candidate plant genotypes meeting a given threshold value for an advancement score, being within a given percentile of the candidate plant genotypes, or a certain number of candidate plant genotypes having the highest or lowest advancement scores.

14. The method of claim 2, wherein the method comprises determining a ranking of the candidate plant genotypes based on the advancement score or an average advancement score for each candidate plant genotype for one, two, or more breeders.

15. The method of claim 2, wherein the advancement score comprises applying a penalty.

16. A computer readable medium having stored thereon instructions to provide candidate plant genotypes recommendations, when executed by a processor (or computing device), cause the processor to perform the steps of claim 1, or both.

17. A computer readable medium having stored thereon instructions to provide candidate plant genotypes recommendations, when executed by a processor (or computing device), cause the processor to perform the steps of claim 2.

18. A system for use in plant breeding comprising:
(a) one or more servers, each of the one or more server storing plant data; and
(b) a computing device communicatively coupled to the one or more servers, the computing device including:
(1) a memory; and
(2) one or more processors configured to perform operations comprising:
(a) obtain data from a plurality of candidate plant genotypes; and
(b) generate an advancement score for each candidate plant genotype from the plurality of candidate plant genotypes using an ensemble of at least two trained machine learning models.

19. A system for use in plant breeding comprising:
(a) one or more servers, each of the one or more server storing plant data; and
(b) a computing device communicatively coupled to the one or more servers, the computing device including:
(1) a memory; and
(2) one or more processors configured to perform operations comprising:
(a) receive into a pretrained deep learning model a plurality of candidate plant genotypes that a breeding target environment is considering along with a breeding target environment token that is considering a plurality of candidate plant genotypes to generate an advancement score for each candidate plant genotype.

20. The system of claim 19, wherein the one or more processors are configured to perform the operations comprising:
(a) obtain one or more training plant genotype representations and one or more representations of breeding target environments associated with the training plant genotype representations;
(b) simultaneously learn one or more associations among training plant genotypes and between training plant genotypes and breeding target environments to produce predicted associations whether training plant genotypes are associated with one another and with a given breeding target environment using a deep learning model implementing self-attention;
(c) evaluate a loss function of the predicted associations among the training plant genotypes and predicted associations among the training plant genotypes and breeding target environments with respect to their true grouping values;
(d) adjust the weights of the self-attention model, and/or the embedding model, and/or the predictive output layer of the tokens to reduce the evaluated loss; and
(e) reiterate steps (a)-(d) until convergence of the loss to a desired value.

21. The system of claim 20, wherein the one or more processors are configured to perform the operations comprising:
(f) obtain by the pre-trained deep learning model implementing self-attention one or more candidate plant genotype representations and one or more representations of breeding target environments associated with the plant genotype representations; and
(g) evaluate a loss function of the predicted advancement score for each of the candidate plant genotypes with respect to their true advancement values; and
(h) adjust the weights of the deep learning self-attention model, the embedding model, the predictive output layer of the tokens to reduce the evaluated loss.

22. The system of claim 19, wherein the one or more processors are configured to perform the operation comprising:
(i) reiterate steps (f)-(h) until convergence of the loss to a desired value.

23. The system of claim 19, wherein the one or more processors are configured to perform the operation comprising:
generate by an advancement score for each candidate plant genotype for a particular environment or region and/or for a particular characteristic/trait.

24. The system of claim 19, wherein the one or more processors are configured to perform the operation comprising:
generate an average advancement score from two or more breeders for each candidate plant genotype.

25. The system of claim 19, wherein the one or more processors are configured to perform the operation comprising:
select one or more candidate plant genotypes based on its advancement score.

26. The system of claim 19, wherein the one or more processors are configured to perform the operation comprising:
select a subset of candidate plant genotypes from a plurality of candidate plant genotypes based on the advancement scores of the candidate plant genotypes meeting a given threshold value for an advancement score, being within a given percentile of the candidate plant genotypes' advancement scores, or a certain number of candidate plant genotypes having the highest or lowest advancement scores.

27. The system of claim 19, wherein the one or more processors are configured to perform the operation comprising:
rank the candidate plant genotypes based on the advancement score or an average advancement score for each candidate plant genotype for one, two, or more breeders.

28. The system of claim 19, wherein the one or more processors are configured to perform the operation comprising:
apply a penalty to the advancement score.

29.-37. (canceled)

38. The method of claim 2, wherein the plant genotype or candidate plant genotype is for a monocot or dicot plant.

39. The method of claim 2, wherein the plant genotype or candidate plant genotype is for a soybean, maize, sorghum, cotton, canola, sunflower, rice, wheat, sugarcane, alfalfa tobacco, barley, cassava, peanuts, millet, oil palm, potatoes, rye, or sugar beet plant.

40. The system of claim 19, wherein the plant genotype or candidate plant genotype is for a monocot or dicot plant.

41. The system of claim 19, wherein the plant genotype or candidate plant genotype is for a soybean, maize, sorghum, cotton, canola, sunflower, rice, wheat, sugarcane, alfalfa tobacco, barley, cassava, peanuts, millet, oil palm, potatoes, rye, or sugar beet plant.
